(19) United States
(12) Patent Application Publication
(10) Pub. No.: US 2024/0338117 A1
(43) Pub. Date: Oct. 10, 2024

(54) BIOLOGICAL PARTICLE SORTING DEVICE, BIOLOGICAL PARTICLE SORTING SYSTEM, AND INFORMATION PROCESSING DEVICE

(71) Applicant: Sony Group Corporation, Tokyo (JP)

(72) Inventors: Koichiro Sato, Kanagawa (JP); Kiyoshi Kagawa, Kanagawa (JP); Shinichi Yoshida, Kanagawa (JP); Muneaki Osawa, Chiba (JP); Ayako Iwase, Kanagawa (JP); Yudai Yanashita, Kanagawa (JP)

(73) Assignee: Sony Group Corporation, Tokyo (JP)

(21) Appl. No.: 18/293,658

(22) PCT Filed: Mar. 17, 2022
(86) PCT No.: PCT/JP2022/012338

§ 371 Date: Jan. 30, 2024

(30) Foreign Application Priority Data
Aug. 13, 2021 (JP) 2021-131976

(51) Int. Cl. G06F 3/0486; G06F 3/04847; G06F 3/0488; G16B 40/10

(52) U.S. Cl. CPC G06F 3/0486; G06F 3/04847; G06F 3/0488; G16B 40/10; G06F 2203/04804

(57) ABSTRACT

A main object of the present disclosure is to improve operability of a biological particle sorting device, particularly to improve operability of a user interface of the device.

The present disclosure provides a biological particle sorting device including a display unit that displays a measurement data display area, in which in a display mode that displays blank spaces in addition to one or more pieces of measurement data in the measurement data display area, in response to a user selecting one piece of movement target measurement data of the one or more pieces of measurement data and one movement destination position in the blank space, the movement target measurement data is displayed at the movement destination position, and after a display position of the movement target measurement data is changed, a position where the movement target measurement data exists is displayed as blank spaces.

### TECHNICAL FIELD

The present disclosure relates to a biological particle sorting device, a biological particle sorting system, and an information processing device. More specifically, the present disclosure relates to a biological particle sorting device, a biological particle sorting system, and an information processing device that display a screen related to control and/or execution of biological particle sorting processing.

### BACKGROUND ART

In flow cytometry, light derived from various fluorescent dyes is analyzed multidimensionally. For the analysis, a large number of graphs and/or tables of statistical values are displayed on the screen of the information processing device, and for example, a gate is set or adjusted. Furthermore, a desired biological particle is sorted using the analysis results. The device that executes the sorting is also called a cell sorter. Furthermore, in recent years, a device for sorting the biological particle in a closed space has also been proposed, and the device is also called a closed sorter.

Several proposals have been made so far for the closed sorters. For example, Patent Document 1 below discloses a microparticle sorting device including a determination unit that determines whether microparticles are sorted, based on light generated by irradiating, with light, the microparticles flowing in a flow channel, in which the determination unit performs a primary sorting determination that determines, based on characteristics of the light, whether the microparticles belong to any one of two or more different microparticle populations, and then, performs a secondary sorting determination that determines, based on a particle constituent ratio specified for the two or more different microparticle populations, whether the microparticles determined to belong to any one of the microparticle populations in the primary sorting determination are sorted.

### CITATION LIST

### Patent Document

Patent Document 1: Japanese Patent Application Laid-Open No. 2020-076736

### SUMMARY OF THE INVENTION

### Problems to be Solved by the Invention

For the sorting processing by the biological particle sorting device, sorting processing conditions such as gate setting are set. The setting is performed via various data displayed on the screen as described above. In order to perform this setting, highly specialized knowledge (for example, knowledge regarding the biological particles and knowledge regarding the sorting device) is often required. In devices such as a flow cytometer and a cell sorter which have been commercially available so far, a user interface thereof is designed on the assumption that a user has such specialized knowledge.

However, for example, research and development as well as manufacturing related to cell therapeutic agents are performed in a clean room in order to avoid contamination. Therefore, in a case where a biological particle sorting device such as a closed sorter is used in a clean room, for example, as illustrated in FIG. 1, it is assumed that a user U operates a biological particle sorting device A in a state of wearing work clothes and gloves for a clean room. The gloves are, for example, lab gloves containing a material such as latex. In a case where such gloves are worn on hands, fine manipulation is often difficult. For example, regarding the touch panel, it is difficult to perform fine operation in a state where the gloves are worn. Therefore, it is desirable to provide the user interface that is easy to use or operate even in such a state.

Therefore, a main object of the present disclosure is to improve operability of the biological particle sorting device, particularly to improve the operability of the user interface of the device.

### Solutions to Problems

The present disclosure is to provide a biological particle sorting device, including:
a display unit that displays a measurement data display area,
in which in a display mode that displays blank spaces in addition to one or more pieces of measurement data in the measurement data display area,
in response to a user selecting one piece of movement target measurement data of the one or more pieces of measurement data and one movement destination position in the blank spaces, the movement target measurement data is displayed at the movement destination position; and
after a display position of the movement target measurement data is changed, a position where the movement target measurement data exists is displayed as the blank spaces.

The biological particle sorting device may be configured to display the one or more pieces of measurement data in a grid pattern in the measurement data display area.

The biological particle sorting device may be configured to display a candidate position image indicating a position where the measurement data can be arranged in the blank spaces.

The biological particle sorting device may be configured not to change an arrangement configuration of measurement data other than the movement target measurement data between before and after the movement of the movement target measurement data to the blank spaces.

The biological particle sorting device has a plurality of display modes, and
the biological particle sorting device may cause the blank spaces to appear in the measurement data display area in response to selection of a display mode that displays the blank spaces from among the plurality of display modes, or
the biological particle sorting device may cause the blank spaces to appear in the measurement data display area in response to the selection of a display mode that displays the blank spaces and selection of movement target measurement data from among the plurality of display modes.

Number of columns at which measurement data can be arranged in the measurement data display area may be set in advance.

The biological particle sorting device may be configured to be able to display a setting panel that changes the number of columns.

The biological particle sorting device may increase the number of columns in response to the selection of a display mode that displays the blank spaces.

The biological particle sorting device may display the increased columns as the blank spaces.

The setting panel may be displayed translucently.

The biological particle sorting device may be configured to be able to display a gate edit panel that edits a gate set in the measurement data, and the gate edit panel may be configured to be able to select a gate to be edited.

The gate edit panel may be configured to be able to select the entire selected gate or a configuration component of the selected gate as an object to be edited.

The biological particle sorting device is configured to be able to display a plot or an axis edit panel that edits a display form and/or an axis of measurement data, and

the plot or axis edit panel may be configured to be able to select an axis to be edited.

The display unit displays, in addition to the measurement data display area, a sorting operation control area displaying a button that controls a sorting operation by the biological particle sorting device, and

the biological particle sorting device may be configured to be able to expand and display data associated with the sorting operation control area on the measurement data display area.

The display unit displays, in addition to the measurement data display area, an operation button area that displays one or more operation buttons used to set sorting conditions, and

the biological particle sorting device may be configured to be able to expand and display the operation button area on the measurement data display area in response to selection of any of the one or more operation buttons.

A button that selects a display mode that displays the blank spaces may be displayed in the operation button area.

The display unit may be configured to facilitate touch input.

Furthermore, the present disclosure is to provide a biological particle sorting system including:
a display unit that displays a measurement data display area
in which in a display mode that displays the blank spaces in addition to one or more pieces of measurement data in the measurement data display area,
in response to a user selecting one piece of movement target measurement data of the one or more pieces of measurement data and one movement destination position in the blank spaces, the movement target measurement data is displayed at the movement destination position; and
after the display position of the movement target measurement data is changed, a position where the movement target measurement data exists is displayed as the blank spaces.

Furthermore, the present disclosure is also to provide an information processing device including:
a display unit that displays a measurement data display area,
in which in a display mode that displays the blank spaces in addition to one or more pieces of measurement data in the measurement data display area,
in response to a user selecting one piece of movement target measurement data of the one or more pieces of measurement data and one movement destination position in the blank spaces, the movement target measurement data is displayed at the movement destination position; and
after the display position of the movement target measurement data is changed, a position where the movement target measurement data exists is displayed as the blank spaces.

### BRIEF DESCRIPTION OF DRAWINGS

FIG. 1 is a schematic diagram illustrating an example of a situation in which a biological particle sorting device is operated.

FIG. 2A is a diagram illustrating a configuration example of a biological sample analyzer of the present disclosure.

FIG. 2B is an example of a block diagram of the biological particle sorting device of the present disclosure.

FIG. 3 is an example of a screen displayed by the biological particle sorting device of the present disclosure.

FIG. 4 is an example of a screen displayed by the biological particle sorting device of the present disclosure, and is a diagram that describes three areas that can be included in the screen.

FIG. 5 is a diagram that describes a first display area.

FIG. 6A is a diagram that describes a second display area.

FIG. 6B is a diagram that describes a scroll bar displayed in the second display area.

FIG. 7 is a diagram that describes a third display area.

FIG. 8 is a diagram that describes an arrangement example of measurement data in the second display area.

FIG. 9A is a diagram that describes an operation of moving measurement data to the blank spaces.

FIG. 9B is a diagram that describes the operation of moving the measurement data to the blank spaces.

FIG. 9C is a diagram that describes the operation of moving the measurement data to the blank spaces.

FIG. 10 is a diagram that describes how to move the measurement data.

FIG. 11A is a diagram that describes the operation of moving the measurement data to the blank spaces.

FIG. 11B is a diagram that describes the operation of moving the measurement data to the blank spaces.

FIG. 11C is a diagram that describes the operation of moving the measurement data to the blank spaces.

FIG. 12A is a diagram that describes the operation of moving the measurement data to a position where other measurement data exists.

FIG. 12B is a diagram that describes the operation of moving the measurement data to the position where the other measurement data exists.

FIG. 12C is a diagram that describes the operation of moving the measurement data to the position where the other measurement data exists.

FIG. 12D is a diagram that describes the operation of moving the measurement data to the position where the other measurement data exists.

FIG. 13A is a diagram that describes an operation of deleting a row or a column including only the blank spaces.

FIG. 13B is a diagram that describes the operation of deleting the row or the column including only the blank spaces.

FIG. 13C is a diagram that describes the operation of deleting the row or the column including only the blank spaces.

FIG. 13D is a diagram that describes the operation of deleting the row or the column including only the blank spaces.

FIG. 14A is a diagram that describes screen transition in which the blank spaces is displayed in response to selection of Plot Arrange View mode.

FIG. 14B is a diagram that describes the screen transition in which the blank spaces is displayed in response to the selection of Plot Arrange View mode.

FIG. 14C is a diagram that describes the screen transition in which the blank spaces is displayed in response to the selection of Plot Arrange View mode.

FIG. 14D is a diagram that describes the screen transition in which the blank spaces is displayed in response to the selection of Plot Arrange View mode.

FIG. 15A is a diagram that describes an example of a display mode for the gate edit panel.

FIG. 15B is a diagram that describes the example of the display mode for the gate edit panel.

FIG. 15C is a diagram that describes the example of the display mode for the gate edit panel.

FIG. 16 is a diagram that describes the configuration example of the gate edit panel.

FIG. 17 is a diagram that describes a drop-down menu in the gate edit panel.

FIG. 18A is a diagram that describes an example of a display mode of a plot/axis edit panel.

FIG. 18B is a diagram that describes the example of the display mode of the plot/axis edit panel.

FIG. 18C is a diagram that describes the example of the display mode of the plot/axis edit panel.

FIG. 19 is a diagram that describes a configuration example of the plot/axis edit panel.

FIG. 20A is a diagram that describes an example of a display method of a worksheet setting panel.

FIG. 20B is a diagram that describes the example of the display method of the worksheet setting panel.

FIG. 20C is a diagram that describes the example of the display method of the worksheet setting panel.

FIG. 20D is a diagram that describes the example of the display method of the worksheet setting panel.

FIG. 21 is a diagram that describes the configuration example of the worksheet setting panel.

FIG. 22A is a diagram illustrating an example of an embodiment in which display contents in the first display area are displayed in superposition with the second display area.

FIG. 22B is a diagram illustrating the example of the embodiment in which the display contents in the first display area are displayed in superposition with the second display area.

FIG. 22C is a diagram illustrating an example of an embodiment in which the display contents in the first display area are displayed in superposition with the second display area.

FIG. 23A is a diagram illustrating an example of an embodiment in which display contents in the third display area are displayed in superposition with the second display area.

FIG. 23B is a diagram illustrating the example of the embodiment in which the display contents in the third display area are displayed in superposition with the second display area.

FIG. 23C is a diagram illustrating the example of the embodiment in which the display contents in the third display area are displayed in superposition with the second display area.

FIG. 24 is a diagram illustrating an example of a biological particle sorting system according to the present disclosure.

FIG. 25 is a diagram that describes a configuration example of the plot/axis edit panel.

### MODE FOR CARRYING OUT THE INVENTION

Hereinafter, preferred modes for carrying out the present disclosure will be described. Note that the embodiments described below show typical embodiments of the present disclosure, and the scope of the present disclosure should not be limited to these embodiments. Note that the present disclosure will be described in the following order.
1. Basic concept of present disclosure
2. First embodiment of present disclosure (biological particle sorting device)
(1) Device configuration example
(2) Example of screen to be displayed
(2-1) Processing condition setting screen
(2-2) Measurement data movement processing (use of blank spaces)
(2-2-1) Measurement data movement processing to blank spaces
(2-2-2) Measurement data movement processing to a position where other measurement data exists
(2-2-3) Processing of deleting rows or columns of blank spaces
(2-2-4) Display method of blank spaces
(2-3) Operation panel
(2-3-1) Gate edit panel
(2-3-2) Plot/axis edit panel
(2-3-3) Measurement data display area setting panel
(2-4) Display processing of superimposing and displaying contents of other display areas on measurement data display area
(2-4-1) Embodiment in which display contents in first display area are displayed in superposition with second display area
(2-4-2) Embodiment in which display contents in third display area are displayed in superposition with second display area
(3) Modification example
3. Second embodiment of present disclosure (biological particle sorting system)

### 1. Basic Concept of Present Disclosure

As described above, in a case where the biological particle sorting device is operated in a clean room, a user who operates the biological particle sorting device generally wears gloves. In a case where such gloves are worn on hands, fine manipulation is often difficult. For example, regarding the touch panel, it is difficult to perform fine operation in a state where the gloves are worn. Therefore, it is desirable to provide the user interface that is easy to use or operate even in such a state.

Furthermore, sorting setting operation screens of many biological particle sorting devices are based on a premise that detailed operations are performed in order for a user having specialized knowledge to perform detailed settings. On the setting operation screen described above, for example, various settings are performed while moving a cursor on a window with a mouse or the like. It is not assumed that such a setting operation screen is subjected to a touch operation, and setting by a touch operation is often difficult.

Furthermore, a plurality of pieces of measurement data (for example, plot data and/or histogram data) is displayed on the screen on the sorting condition screen (for example, the gate setting screen) of the biological particle sorting device. It is desirable that the arrangement of the plurality of pieces of measurement data can be freely changed, but if the arrangement of the plurality of pieces of measurement data is significantly changed between before and after the change of the arrangement, there is a case where it is difficult for the user who has set the sorting conditions to use the plurality of pieces of measurement data. For example, in a case where the gate setting has already been completed for some pieces of plot data among the plurality of pieces of plot data, there is a case where it is undesirable for the user to change the arrangement of such pieces of plot data for which the gate setting has been completed.

A biological particle sorting device according to the present disclosure includes a display unit that displays a measurement data display area. In a display mode in which the biological particle sorting device displays the blank spaces in addition to one or more pieces of measurement data in the measurement data display area, the biological particle sorting device displays the movement target measurement data at the movement destination position in response to the user selecting one movement target measurement data of the one or more pieces of measurement data and one movement destination position in the blank spaces, and displays a position where the movement target measurement data existed as blank spaces after the display position of the movement target measurement data is changed.

By changing the display position of the measurement data as such, for example, movement of the measurement data by the touch operation can be easily performed. Furthermore, it is possible to reduce a necessity of changing the arrangement of other measurement data before and after a change of the display position. Therefore, it is possible to prevent the user from being confused by an arrangement change of the measurement data. In addition, by displaying the position where the movement target measurement data exists as the blank spaces, it is easy to grasp the measurement data whose arrangement has been changed before and after the movement and the measurement data whose arrangement has been maintained before and after the movement.

The display unit may be included as a configuration component of an information processing device that controls the biological particle sorting device. In other words, the present disclosure also provides an information processing device including the display unit.

Furthermore, the display unit may be included as a configuration component of the biological particle sorting system. For example, the display unit may be included as a configuration component of the biological particle sorting system in which one or more biological particle sorting devices and one or more information processing devices are connected via a network. In such a system, the display unit may be included in the information processing device or may be included in the biological particle sorting devices.

Hereinafter, first, a configuration example of a device will be described, and next, an example of a screen displayed according to the present disclosure will be described.

### 2. First Embodiment of Present Disclosure (Biological Particle Sorting Device)

### (1) Configuration Example of Device

The biological particle sorting device according to the present disclosure may be configured as a biological sample analyzer as described below.

FIG. 2A illustrates the configuration example of the biological sample analyzer of the present disclosure. A biological sample analyzer 6100 shown in FIG. 2A includes: a light irradiation unit 6101 that irradiates a biological sample S flowing in a flow channel C with light; a detection unit 6102 that detects light generated by irradiating the biological sample S with light; and an information processing unit 6103 that processes information associated with the light detected by the detection unit. The biological sample analyzer 6100 is a flow cytometer or an imaging cytometer, for example. The biological sample analyzer 6100 may include a sorting unit 6104 that sorts out specific biological particles P in a biological sample. The biological sample analyzer 6100 including the sorting unit is a cell sorter, for example.

### (Biological Sample)

The biological sample S may be a liquid sample containing biological particles. The biological particles are cells or non-cellular biological particles, for example. The cells may be living cells, and more specific examples thereof include blood cells such as erythrocytes and leukocytes, and germ cells such as sperms and fertilized eggs. Also, the cells may be those directly collected from a sample such as whole blood, or may be cultured cells obtained after culturing. The non-cellular biological particles are extracellular vesicles, or particularly, exosomes and microvesicles, for example. The biological particles may be labeled with one or more labeling substances (such as a dye (particularly, a fluorescent dye) and a fluorochrome-labeled antibody). Note that particles other than biological particles may be analyzed by the biological sample analyzer of the present disclosure, and beads or the like may be analyzed for calibration or the like.

### (Flow Channel)

The flow channel C is designed so that a flow of the biological sample S is formed. In particular, the flow channel C may be designed so that a flow in which the biological particles contained in the biological sample are aligned substantially in one row is formed. The flow channel structure including the flow channel C may be designed so that a laminar flow is formed. In particular, the flow channel structure is designed so that a laminar flow in which the flow of the biological sample (a sample flow) is surrounded by the flow of a sheath liquid is formed. The design of the flow channel structure may be appropriately selected by a person skilled in the art, or a known one may be adopted. The flow channel C may be formed in a flow channel structure such as a microchip (a chip having a flow channel on the order of micrometers) or a flow cell. The width of the flow channel C is 1 mm or smaller, or particularly, may be not smaller than 10 μm and not greater than 1 mm. The flow channel C and the flow channel structure including the flow channel C may be made of a material such as plastic or glass.

The biological sample analyzer of the present disclosure is designed so that the biological sample flowing in the flow channel C, or particularly, the biological particles in the biological sample are irradiated with light from the light irradiation unit 6101. The biological sample analyzer of the present disclosure may be designed so that the irradiation point of light on the biological sample is located in the flow channel structure in which the flow channel C is formed, or may be designed so that the irradiation point is located outside the flow channel structure. An example of the former case may be a configuration in which the light is emitted onto the flow channel C in a microchip or a flow cell. In the latter case, the biological particles after exiting the flow channel structure (particularly, the nozzle portion thereof) may be irradiated with the light, and a flow cytometer of a jet-in-air type can be adopted, for example.

### (Light Irradiation Unit)

The light irradiation unit 6101 includes a light source unit that emits light, and a light guide optical system that guides the light to the irradiation point. The light source unit includes one or more light sources. The type of the light source(s) is a laser light source or an LED, for example. The wavelength of light to be emitted from each light source may be any wavelength of ultraviolet light, visible light, and infrared light. The light guide optical system includes optical components such as beam splitters, mirrors, or optical fibers, for example. The light guide optical system may also include a lens group for condensing light, and includes an objective lens, for example. There may be one or more irradiation points at which the biological sample and light intersect. The light irradiation unit 6101 may be designed to collect light emitted onto one irradiation point from one light source or different light sources.

### (Detection Unit)

The detection unit 6102 includes at least one photodetector that detects light generated by emitting light onto biological particles. The light to be detected may be fluorescence or scattered light (such as one or more of the following: forward scattered light, backscattered light, and side scattered light), for example. Each photodetector includes one or more light receiving elements, and has a light receiving element array, for example. Each photodetector may include one or more photomultiplier tubes (PMTs) and/or photodiodes such as APDs and MPPCs, as the light receiving elements. The photodetector includes a PMT array in which a plurality of PMTs is arranged in a one-dimensional direction, for example. The detection unit 6102 may also include an image sensor such as a CCD or a CMOS. With the image sensor, the detection unit 6102 can acquire an image (such as a bright-field image, a dark-field image, or a fluorescent image, for example) of biological particles.

The detection unit 6102 includes a detection optical system that causes light of a predetermined detection wavelength to reach the corresponding photodetector. The detection optical system includes a spectroscopic unit such as a prism or a diffraction grating, or a wavelength separation unit such as a dichroic mirror or an optical filter. The detection optical system is designed to disperse the light generated by light irradiation to biological particles, for example, and detect the dispersed light with a larger number of photodetectors than the number of fluorescent dyes with which the biological particles are labeled. A flow cytometer including such a detection optical system is called a spectral flow cytometer. Further, the detection optical system is designed to separate the light corresponding to the fluorescence wavelength band of a specific fluorescent dye from the light generated by the light irradiation to the biological particles, for example, and cause the corresponding photodetector to detect the separated light.

The detection unit 6102 may also include a signal processing unit that converts an electrical signal obtained by a photodetector into a digital signal. The signal processing unit may include an A/D converter as a device that performs the conversion. The digital signal obtained by the conversion performed by the signal processing unit can be transmitted to the information processing unit 6103. The digital signal can be handled as data related to light (hereinafter, also referred to as “light data”) by the information processing unit 6103. The light data may be light data including fluorescence data, for example. More specifically, the light data may be data of light intensity, and the light intensity may be light intensity data of light including fluorescence (the light intensity data may include feature quantities such as area, height, and width).

### (Information Processing Unit)

The information processing unit 6103 includes a processing unit that performs processing of various kinds of data (light data, for example), and a storage unit that stores various kinds of data, for example. In a case where the processing unit acquires the light data corresponding to a fluorescent dye from the detection unit 6102, the processing unit can perform fluorescence leakage correction (a compensation process) on the light intensity data. In the case of a spectral flow cytometer, the processing unit also performs a fluorescence separation process on the light data, and acquires the light intensity data corresponding to the fluorescent dye. The fluorescence separation process may be performed by an unmixing method disclosed in JP 2011-232259 A, for example. In a case where the detection unit 6102 includes an image sensor, the processing unit may acquire morphological information about the biological particles, on the basis of an image acquired by the image sensor. The storage unit may be designed to be capable of storing the acquired light data. The storage unit may be designed to be capable of further storing spectral reference data to be used in the unmixing process.

In a case where the biological sample analyzer 6100 includes the sorting unit 6104 described later, the information processing unit 6103 can determine whether to sort the biological particles, on the basis of the light data and/or the morphological information. The information processing unit 6103 then controls the sorting unit 6104 on the basis of the result of the determination, and the biological particles can be sorted by the sorting unit 6104.

The information processing unit 6103 may be designed to be capable of outputting various kinds of data (such as light data and images, for example). For example, the information processing unit 6103 can output various kinds of data (such as a two-dimensional plot or a spectrum plot, for example) generated on the basis of the light data. The information processing unit 6103 may also be designed to be capable of accepting inputs of various kinds of data, and accepts a gating process on a plot by a user, for example. The information processing unit 6103 may include an output unit (such as a display, for example) or an input unit (such as a keyboard, for example) for performing the output or the input.

The information processing unit 6103 may be designed as a general-purpose computer, and may be designed as an information processing device that includes a CPU, a RAM, and a ROM, for example. The information processing unit 6103 may be included in the housing in which the light irradiation unit 6101 and the detection unit 6102 are included, or may be located outside the housing. Further, the various processes or functions to be executed by the information processing unit 6103 may be realized by a server computer or a cloud connected via a network.

### (Sorting Unit)

The sorting unit 6104 performs sorting of biological particles, in accordance with the result of determination performed by the information processing unit 6103. The sorting method may be a method by which droplets containing biological particles are generated by vibration, electric charges are applied to the droplets to be sorted, and the traveling direction of the droplets is controlled by an electrode. The sorting method may be a method for sorting by controlling the traveling direction of biological particles in the flow channel structure. The flow channel structure has a control mechanism based on pressure (injection or suction) or electric charge, for example. An example of the flow channel structure may be a chip (the chip disclosed in JP 2020-76736 A, for example) that has a flow channel structure in which the flow channel C branches into a recovery flow channel and a waste liquid flow channel on the downstream side, and specific biological particles are collected in the recovery flow channel.

### (2) Example of Screen to be Displayed

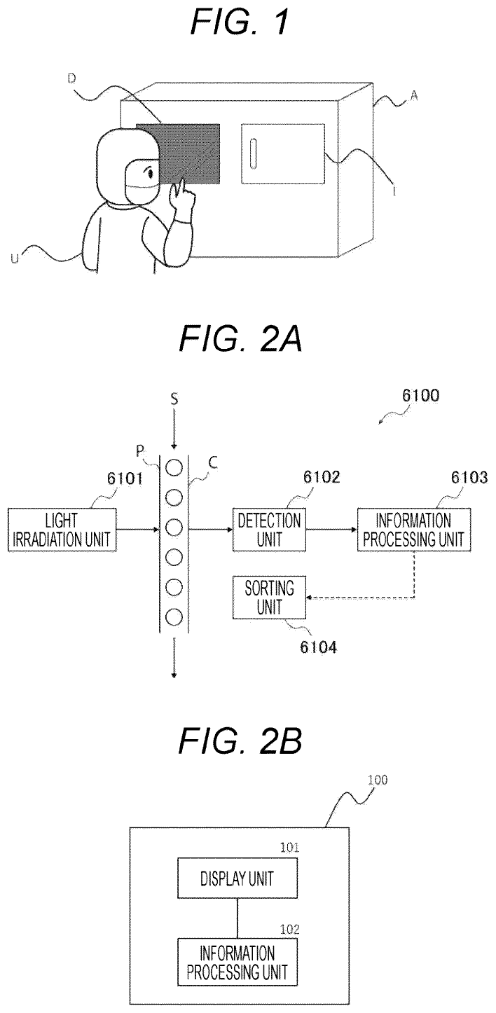

The biological particle sorting device according to the present disclosure may have a display unit D capable of touch input, for example, as illustrated in FIG. 1. As illustrated in FIG. 1, the display unit may be arranged on any surface of the housing of the device, particularly on a side surface, and for example, the screen of the display unit may be arranged at a position where the user standing (or sitting) near the device can perform the touch input. A processing condition setting screen to be described later may be displayed on the display unit. Furthermore, as illustrated in FIG. 1, the biological particle sorting device according to the present disclosure may have an introduction section I for introducing a biological particle-containing sample into the device. For example, a container containing the sample is put into the device from the introduction unit, and the container is connected to a predetermined sample line. Then, after the connection, the sorting processing may be executed.

FIG. 2B illustrates the example of the block diagram of the biological particle sorting device according to the present disclosure. As illustrated in FIG. 2B, a biological particle sorting device 100 according to the present disclosure includes a display unit 101. The biological particle sorting device 100 may further include an information processing unit 102.

The display unit 101 outputs a screen in accordance with the present disclosure. In particular, the display unit 101 displays the processing condition setting screen for receiving input of sorting condition data related to the sorting processing of the biological particle-containing sample. Note that the sorting processing may not be performed, and the processing condition setting screen for performing only analysis may be displayed. The screen displayed by the display unit 101 may be a screen based on data transmitted from the information processing unit 102.

The display unit 101 may include a display device known in the art. For example, the display unit 101 includes a display device having a square screen, particularly a rectangular screen, more particularly a horizontally long rectangular screen. A size of the screen may be, for example, 10 inches or more, preferably 11 inches or more, and more preferably 12 inches or more. An upper limit of the size of the screen may not be particularly set, but may be, for example, 30 inches or less, 25 inches or less, 22 inches or less, or 20 inches or less. Such a screen size is suitable for display of a screen displayed in each mode described below.

The information processing unit 102 causes the display unit 101 to display a screen in accordance with the present disclosure. The information processing unit 102 is configured to transmit data that displays the screen to the display unit 101. The information processing unit 102 may be the information processing unit 6103 in (1) described above, but may be an information processing unit prepared separately. Furthermore, the biological particle sorting device 100 may include the light irradiation unit 6101, the detection unit 6102, and the sorting unit 6104 in (1) described above in addition to the display unit 101 and the information processing unit 102.

The biological particle sorting device 100 (particularly, the information processing unit 102) receives an input of the sorting condition data related to the biological particle sorting process. In order to receive the input of the sorting condition data, the display unit 101 may be capable of touch input.

The information processing unit 102 causes the display unit 101 to display the processing condition setting screen. The information processing unit 102 receives an input of the sorting condition data via the processing condition setting screen.

Note that the processing condition setting screen may be displayed on another display device instead of the display unit 101 provided in the biological particle sorting device 100. For example, the biological particle sorting device 100 may display the screen on a display device or an information processing device connected to the biological particle sorting device 100 in a wired or wireless manner via a network.

Hereinafter, a screen displayed by the biological particle sorting device 100 according to the present disclosure will be described. Hereinafter, first, the configuration example of the screen will be described, and next, a display control according to the present disclosure will be described.

### (2-1) Processing Condition Setting Screen

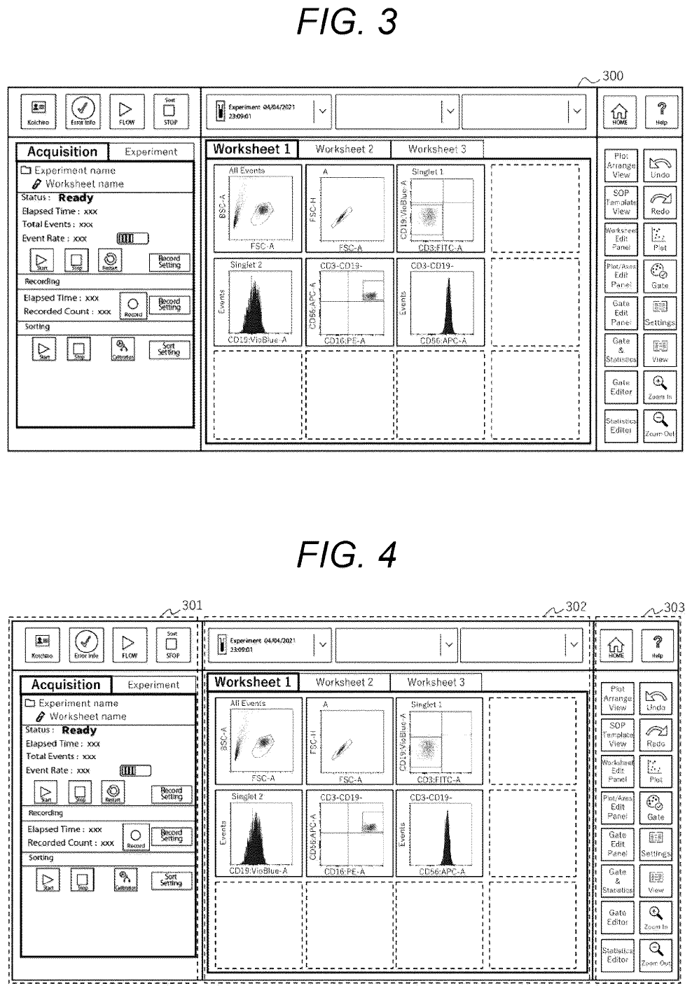

The biological particle sorting device 100 displays, for example, a screen as illustrated in FIG. 3 on the display unit. A screen 300 illustrated in FIG. 3 is a screen for setting the sorting conditions for the biological particle sorting process. As indicated by a broken line in FIG. 4, the screen includes a sorting operation control area (hereinafter also referred to as a “first display area”) 301 displaying buttons that controls (particularly executing) a sorting operation by the biological particle sorting device 100, a measurement data display area (hereinafter also referred to as a “second display area”) 302 displaying one or more pieces of measurement data used to set the sorting conditions, and an operation button area (hereinafter also referred to as a “third display area”) 303 displaying one or more operation buttons used to set the sorting conditions (for example, gate information). The operation button may include a call button that displays various operation panels for setting the sorting conditions.

As shown in these drawings, the first display area, the second display area, and the third display area are arranged on a left side, a center, and a right side of the processing condition setting screen, respectively. These three areas may be arranged in this order from the left in this manner.

Alternatively, the first display area, the second display area, and the third display area may be arranged on a right side, a center, and a left side of the processing condition setting screen, respectively. These three areas may be arranged in this order from the right in this manner.

Note that arrangement of these three areas is not limited to that described above, and for example, may be arranged in order from the top, or may be arranged in order from the bottom.

In order to display more measurement data more largely, the second display area may be displayed to occupy the largest area of the screen 300.

There is a case where the sorting conditions are required to be set in detail. For example, it is necessary to confirm detailed measurement data, or it is necessary to display a large number of plot data and/or histograms on one screen. As described above, by providing the three areas, a large second display area can be secured, which makes it easier for the user to confirm the measurement data.

In order to improve the operability, in a case where the first display area is arranged on the left side of the processing condition setting screen and the third display area is arranged on the right side of the processing condition setting screen, the first display area may be arranged so as to be in contact with a left end of the processing condition setting screen, and the third display area may be arranged so as to be in contact with a right end of the processing condition setting screen.

Conversely, in a case where the first display area is arranged on the right side of the processing condition setting screen and the third display area is arranged on the left side of the processing condition setting screen, the first display area may be arranged so as to be in contact with the right end of the processing condition setting screen, and the third display area may be arranged so as to be in contact with the left end of the processing condition setting screen.

Since the first display area and the third display area are in contact with edges of the screen, it is possible to prevent an unintended touch from occurring during the touch input.

The arrangement of the three areas described above may be changeable. For example, the biological particle sorting device may be configured to change the screen from a state in which the first display area is arranged on the right side and the third display area is arranged on the left side to a state in which a former area is arranged on the left side and a latter area is arranged on the right side. Furthermore, the screen may be configured to be changeable so as to be opposite thereto. Therefore, it is possible to cope with the user's dominant hand or convenience, and the operability can be improved.

### (First Display Area)

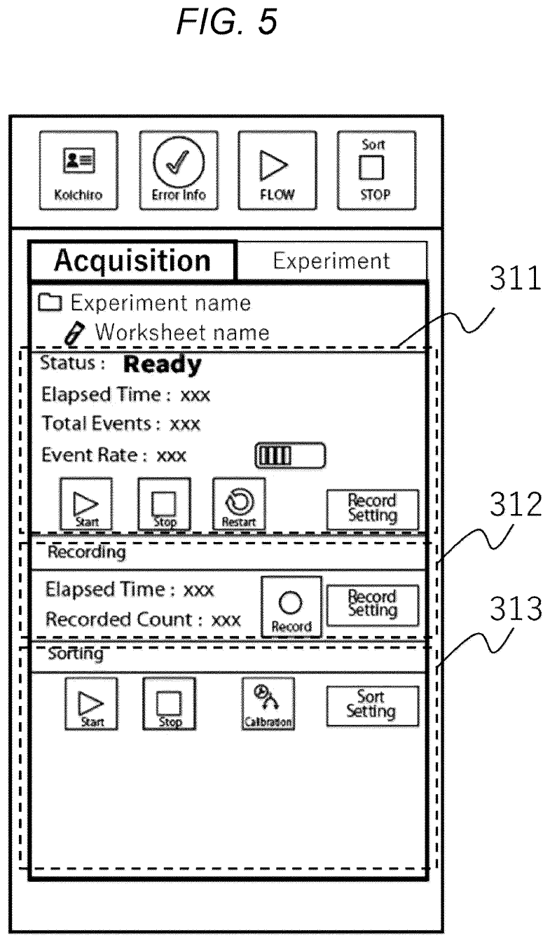

A first display area 300 will be described below with reference to FIG. 5. As illustrated in FIG. 5, the first display area includes an area 311 including one or more buttons that controls flowing of the biological particle-containing sample into the device (particularly, in a microchip on which sorting is performed), an area 312 including one or more buttons that controls recording of a detection result by the detection unit, and an area 313 including one or more buttons that controls the sorting operation. Items displayed in these areas and the buttons arranged in these areas may be appropriately selected by a person skilled in the art.

For example, the area 311 may include a start button (Start button) for starting the flow of the biological particle-containing sample into the device, a button (Stop button) for stopping the flow of the biological particle-containing sample into the device, and the like. Furthermore, in the area 311, an elapsed time of period (Elapsed Time) from a start of flowing of the biological particle-containing sample into the device, the number of detected events (Total Events), an event rate (Event rate), and the like may be displayed.

The area 312 may include a button (a record button) that controls recording of data of the particle detected by the detection unit. Furthermore, in the area 312, the elapsed time of period from the start of the recording (Elapsed Time) and the number of recorded events (Recorded Count) may be displayed.

The area 313 may include a start button (Start button) for starting the biological particle sorting process, a button (Stop button) for stopping the sorting, and the like. Furthermore, a button for executing calibration for sorting may be displayed in the area 313. Furthermore, although not illustrated in FIG. 5, data (for example, the elapsed time) indicating a status of sorting and the like may be displayed in the area 313.

Furthermore, as illustrated in FIG. 5, in the first display area, for example, a name of an experiment file (Experiment name) and a name of a worksheet (Worksheet name) for specifying the sorting conditions may be displayed. The worksheet is, for example, data including the measurement data and the sorting condition data (for example, the gate information). The biological particle sorting device 100 is configured so that the user can perform the gate setting for the measurement data on the worksheet. Furthermore, the biological particle sorting device 100 is configured to be able to sort particles based on the gate set on the worksheet.

Furthermore, as illustrated in FIG. 5, a button that displays a user property, a button that displays error information, a button for starting or ending the sorting process, and the like may be displayed in the first display area, but these buttons may be displayed in other areas.

### (Second Display Area)

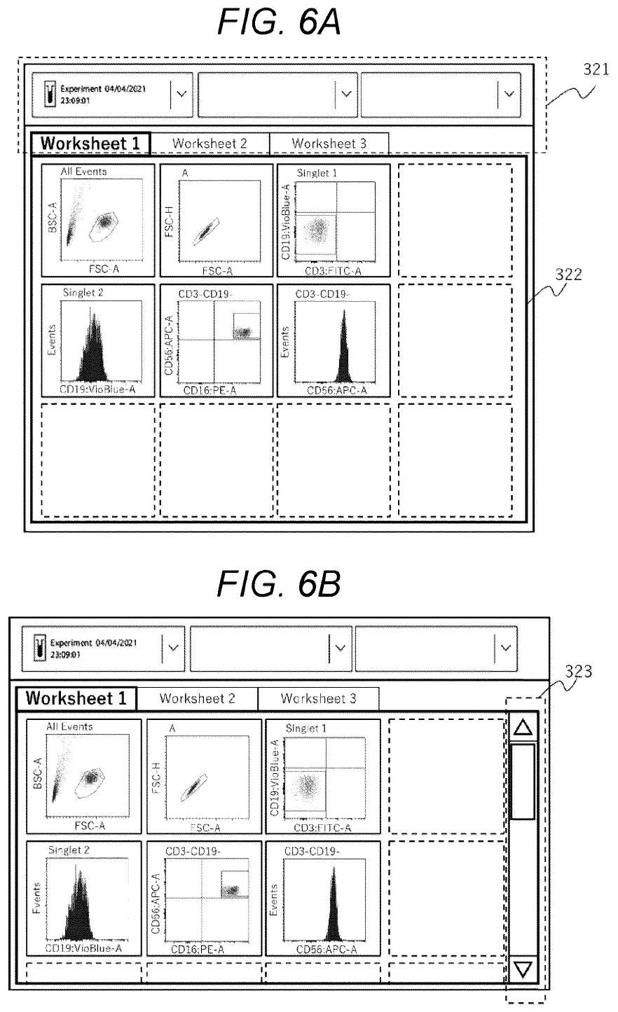

The second display area will be described below with reference to FIG. 6A. As illustrated in FIG. 6A, one or more pieces of measurement data are displayed in the second display area. The one or more pieces of measurement data are data generated by the information processing unit based on the detection result by the detection unit. Each piece of measurement data may be, for example, the histogram data or the plot data (for example, density plot data, dot plot data, contour plot data, and the like). A specific configuration of the measurement data may be appropriately selected by the user.

In FIG. 6A, the second display area is divided into an area 321 that displays the experiment file name and the worksheet name and an area 322 that displays the measurement data, but the configuration of the second display area is not limited to that illustrated in FIG. 6A.

In FIG. 6A, a worksheet named “Worksheet 1” is selected, and six pieces of measurement data included in the worksheet are displayed. In a first row, all three pieces of measurement data are the plot data, and a gate is set for each piece of plot data. Three pieces of measurement data are also displayed in a second row, two of which are histogram data and one of which is the plot data.

As illustrated in the area 321 in FIG. 6A, an experimental file having a file name “Experiment Apr. 4, 2021 23:09:01” is opened, and it is indicated that the experimental file includes a plurality of worksheets (Worksheet 1, Worksheet 2, and Worksheet 3). The plurality of worksheets may be switched between an active state and an inactive state by selecting a tab. In FIG. 6A, a tab of the Worksheet 1 is selected, and the Worksheet 1 is activated.

Preferably, the biological particle sorting device 100 displays the one or more measurement data in the grid pattern. In other words, the one or more pieces of measurement data may be displayed so as to form one or more rows and one or more columns.

For example, in FIG. 6A, the area 322 can arrange the measurement data in a grid pattern of 3 rows and 4 columns. The number of columns of measurement data in the area is four, that is, four pieces of measurement data can be arranged in a row. The number of columns of the measurement data in the same area may be set in advance. Furthermore, the setting of the number of columns of the measurement data in the same area may be changeable by the user. Therefore, it is possible to display a measurement data group in an arrangement that is easy for each user to use.

Furthermore, the number of rows of the measurement data in the area is three, and three pieces of measurement data can be arranged in one column. For example, in a first column from the left, two pieces of measurement data and one blank spaces are arranged. Note that although three pieces of measurement data are displayed as capable of being arranged per column in FIG. 6A, the number of rows may be increased or decreased according to the number of pieces of measurement data.

In the present disclosure, the biological particle sorting device 100 may be capable of setting the number of columns of measurement data displayed in the measurement data display area. Then, the biological particle sorting device 100 may be configured to increase or decrease the number of rows of the measurement data according to the number of measurement data while maintaining the set number of columns.

Furthermore, the biological particle sorting device 100 may be configured to display a scroll bar 323 as illustrated in FIG. 6B, for example, in a case where a row that cannot be displayed is generated in the screen as the number of rows increases. By operating the scroll bar, it is possible to cope with an increase in the number of rows. Note that an increase in the number of columns may also be handled by displaying the scroll bar.

In FIG. 6A, the blank spaces are displayed in a rightmost column and a lowermost row, but these blank spaces may be displayed in a case where a predetermined display mode is selected, and may not be displayed in a case where another display mode is selected.

### (Third Display Area)

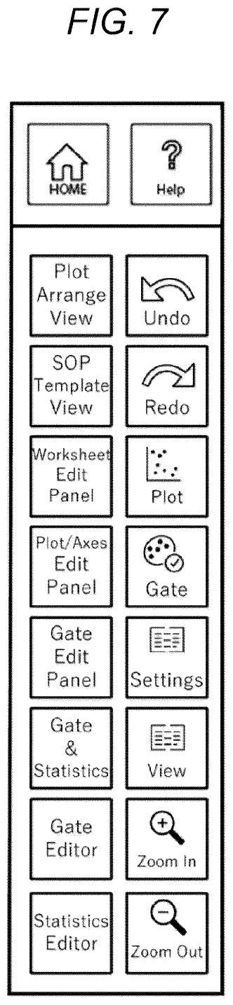

The third display area will be described below with reference to FIG. 7. As illustrated in FIG. 7, a plurality of buttons is displayed in the third display area.

These operation buttons indicate one or more operation buttons to be used for setting the sorting conditions (The sorting conditions include, for example, the gate information. A case where the sorting is not performed is also referred to as an analysis condition). More specifically, these operation buttons may include, in particular, an operation button for setting the gate for the measurement data in the second display area or an operation button that edits the measurement data itself.

These operation buttons may include one or more call buttons that displays various operation panels for setting the sorting conditions.

Examples of the operation buttons are, for example, a Redo button, an Undo button, a zoom-in button, and a zoom-out button as illustrated in FIG. 7. Furthermore, examples of the call button may be a Plot button, a Gate button, a View button, and a Settings button as illustrated in FIG. 7. When these buttons are tapped, a plot operation panel, a gate operation panel, a display mode operation panel, and a setting operation panel may each be displayed in the processing condition setting operation screen.

Furthermore, a button that changes the display mode in the second display area may be displayed in the third display area. For example, the biological particle sorting device 100 changes the second display area to a Plot arrange view mode in response to selection (particularly tapping) of a Plot arrange view button in the third display area by the user. In the Plot arrange view mode, the biological particle sorting device 100 receives a change in arrangement of the measurement data (for example, the plot data and/or the histogram data) displayed in the second display area. This mode is also referred to as a data arrangement adjustment mode. As described above, a button that selects the display mode that displays blank spaces may be displayed in the third display area (the operation button area).

In a preferred embodiment, the biological particle sorting device 100 makes the blank spaces in which the measurement data can be arranged appeared in the measurement data display area in response to tapping of the Plot arrange view button. Before the Plot arrange view button is tapped, the blank spaces may not be displayed in the measurement data display area. Then, according to the tap, for example, as illustrated in FIG. 3, the blank spaces are added to form the rightmost column and/or the lowermost row in the measurement data display area.

The call button may display another operation button in the third display area. For example, a Plot Arrange View mode selection button and other View mode selection buttons to be described later may be displayed in the third display area in response to the selection of the View button. For example, in an initial state, the biological particle sorting device 100 may display only a button group arranged in a right column among the button groups illustrated in FIG. 7. Then, in response to the selection of the View button, a button group arranged in a left column of the button group illustrated in FIG. 7 may be displayed in the third display area. Note that in the present description, the View mode is also referred to as the display mode. The biological particle sorting device of the present disclosure may have the plurality of display modes, and may be configured such that the user can select any one of the display modes.

Furthermore, in response to the selection of the Settings button, a Worksheet Settings button and other Settings buttons to be described later may be displayed in the third display area.

### (2-2) Measurement Data Movement Processing (Use of Blank Spaces)

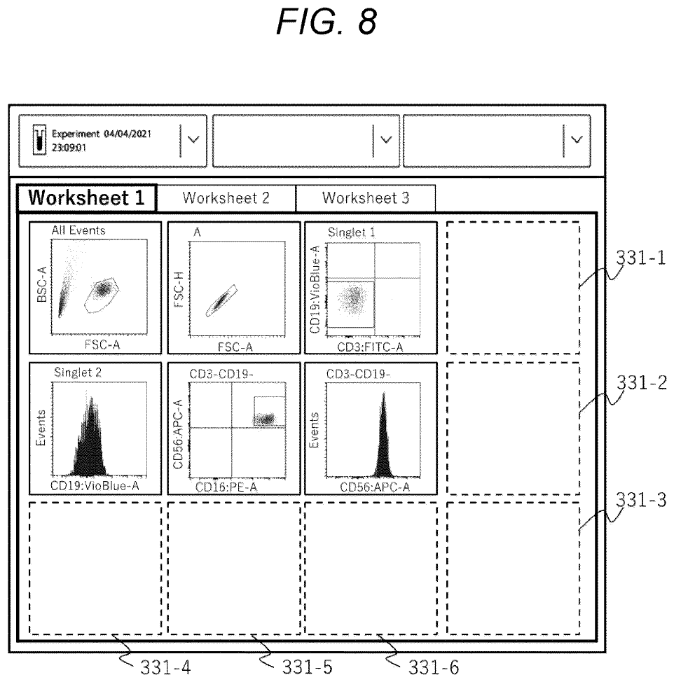

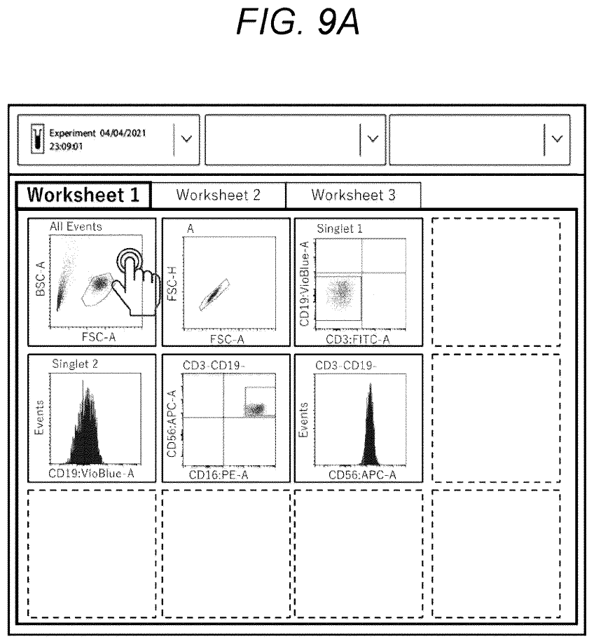

The biological particle sorting device 100 displays measurement data (for example, the histogram data, the plot data, or the like) in the second display area in the Plot arrange view mode. These pieces of measurement data may be arranged in a grid pattern as illustrated in FIG. 8. The arrangement of the measurement data can be changed by a user operation. The Plot arrange view mode is an example of “in a display mode that displays the blank spaces in addition to the one or more pieces of measurement data in the measurement data display area” in the present disclosure.

### (Configuration of Blank Spaces)

As illustrated in FIG. 8, the blank spaces in which the measurement data can be arranged is displayed in the second display area.

As indicated by reference numerals 331-1 to 331-3 in FIG. 8, the blank spaces may be provided so that one column of measurement data can be arranged on the rightmost side in the second display area. Conversely, one column of measurement data may be provided to be capable of being arranged on the leftmost side in the second display area. Although the blank spaces for two or more columns of measurement data may be added, the blank spaces introduced as a column are preferably spaces for one column of measurement data in order to display the already displayed measurement data in a larger size.

Furthermore, as indicated by reference numerals 331-3 to 331-6 in FIG. 8, the blank spaces may be provided so that one row of measurement data can be arranged at the bottom in the second display area. Conversely, one row of measurement data may be arranged at the uppermost stage in the second display area. The blank spaces for two or more rows of measurement data may be added, but the blank spaces introduced as a row is preferably spaces for one row of measurement data in order to display the already displayed measurement data in a larger size.

In the blank spaces, the candidate position image indicating a position where the measurement data can be arranged may be displayed. For example, as indicated by reference numerals 331-1 to 331-6 in FIG. 8, a space in which the measurement data is arranged may be displayed by a dashed polygon (particularly, a quadrangle). Alternatively, the candidate position image may be a colored polygon, a flashing polygon, or the like. Furthermore, an arbitrary mark indicating the candidate position may be displayed. As described above, the biological particle sorting device 100 may be configured to display the candidate position image indicating a position where the measurement data can be arranged in the blank spaces. With such a candidate position image, it is easy to confirm where the movement destination position of the measurement data is, and furthermore, it is easy to predict the state in the second display area after the movement.

### (2-2-1) Measurement Data Movement Processing to Blank Spaces

### (Movement of Measurement Data to Blank Spaces by Tap Operation)

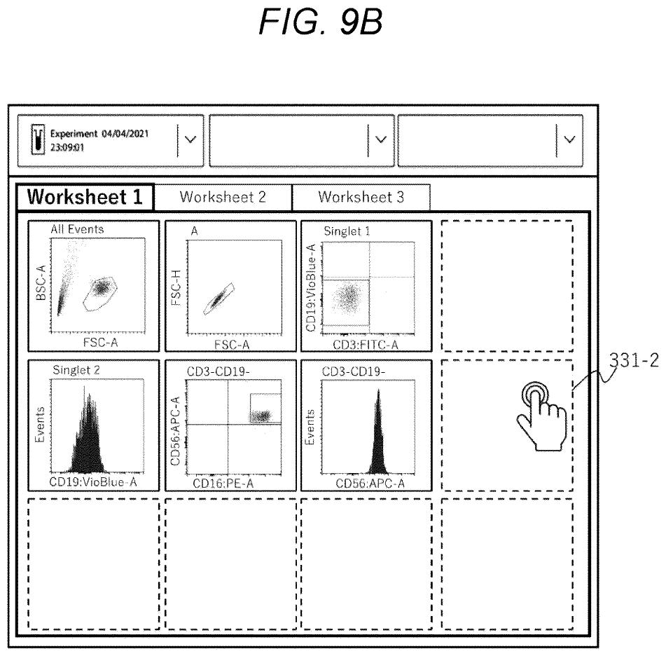

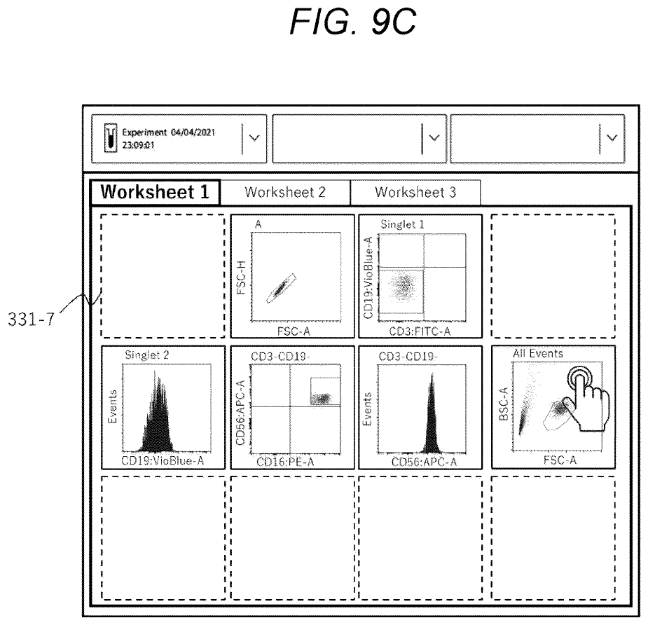

The operation of moving the measurement data to the blank spaces will be described with reference to FIGS. 9A to C. In the measurement data display area illustrated in FIGS. 9A to C, the six pieces of measurement data are displayed. Among them, as illustrated in FIG. 9A, the user taps upper left plot data as the movement target measurement data. Next, as illustrated in FIG. 9B, the user taps blank space 331-2 as the movement destination of the movement target measurement data. In response to these two taps, as illustrated in FIG. 9C, the biological particle sorting device 100 displays the movement target measurement data in the tapped blank spaces. After the display position of the movement target measurement data is changed, the biological particle sorting device 100 displays the position where the movement target measurement data existed as blank space 331-7.

Furthermore, arrangement positions of other measurement data are not changed before and after such movement. In other words, the biological particle sorting device 100 does not change the arrangement configuration of the measurement data other than the movement target measurement data between before and after the movement of the movement target measurement data to the blank spaces. Such a change in the display position of the measurement data can be easily executed even by, for example, a tap operation. Furthermore, since the blank spaces are used, the arrangement of other measurement data does not need to be changed before and after the change of the display position, and therefore, this can prevent the user from being confused by the change of the arrangement of the measurement data. In addition, by displaying the position where the movement target measurement data exists as the blank spaces, it is easy to grasp the measurement data whose arrangement has been changed before and after the movement and the measurement data whose arrangement has been maintained before and after the movement.

### (Method of Moving Measurement Data to Blank Spaces)

As described above, the biological particle sorting device 100 displays the movement target measurement data at the movement destination position in response to the user selecting the movement target measurement data and selecting the movement destination position. When the movement target measurement data is moved from the position before the movement to the movement destination position, the state in which the movement target measurement data is moved may be displayed, or the state may not be displayed.

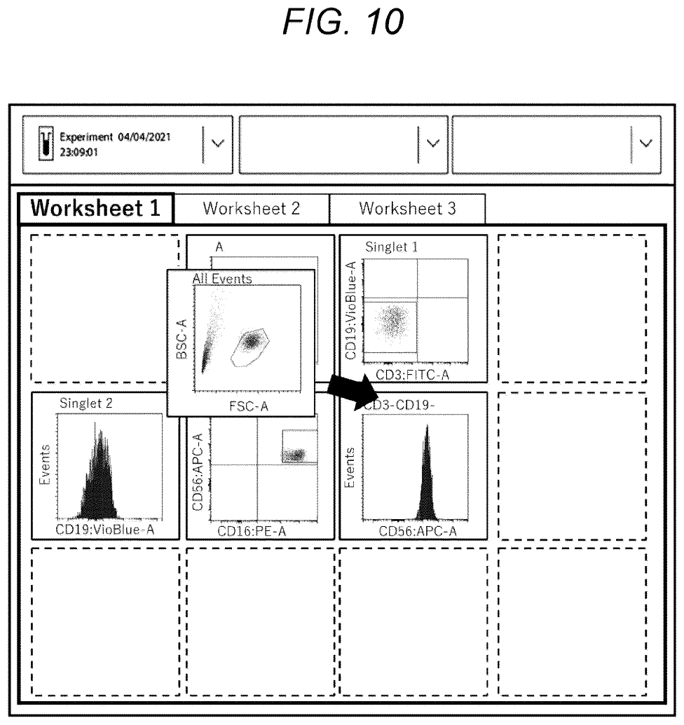

In a former case, for example, as illustrated in FIG. 10, in response to the user selecting the movement target measurement data and selecting the movement destination position, the biological particle sorting device 100 displays, for example, a state in which the movement target measurement data existing at the position before the movement is moving to the movement destination position. Therefore, the user can visually recognize a movement state and easily confirm the movement destination.

In a latter case, in response to the user selecting the movement target measurement data and selecting the movement destination position, the biological particle sorting device 100 may delete the movement target measurement data existing at a position before the movement, change the position to the blank spaces, and cause the movement target measurement data to appear at the movement destination position. Therefore, the time that displays a moving state can be omitted as compared with the former case. For example, image switching from FIG. 9B to FIG. 9C occurs simultaneously with performing the tap shown in FIG. 9B.

### (Movement of Measurement Data to Blank Spaces by Drag and Drop Operations)

Although the embodiment in which the measurement data is moved by the tap operation has been described above, in the present disclosure, the measurement data may be moved by drag and drop operations. In other words, the movement target measurement data may be selected and moved by the drag operation, and then, the movement destination position may be selected by the drop operation. This moving method will be described below.

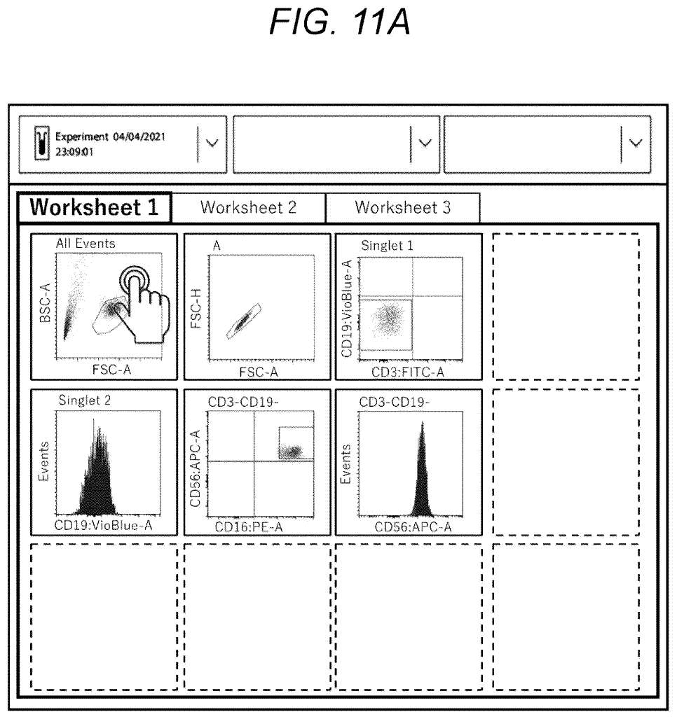

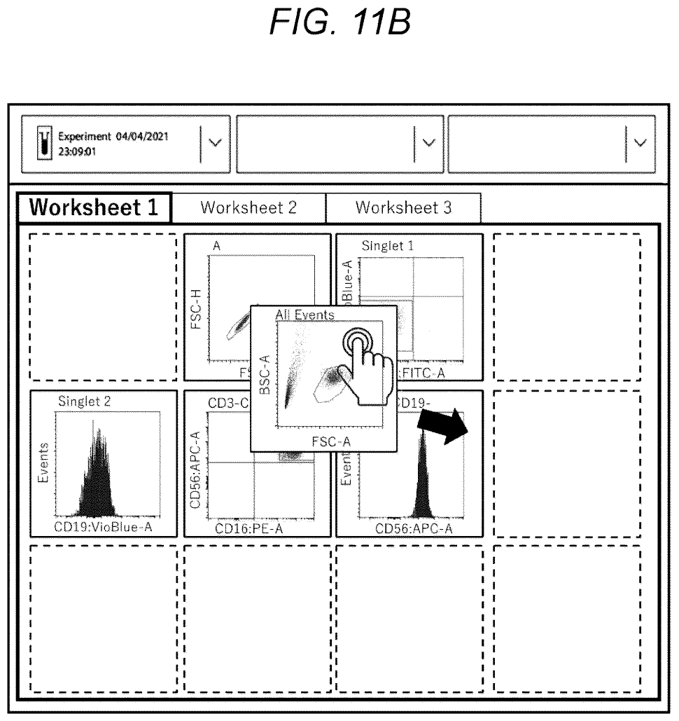

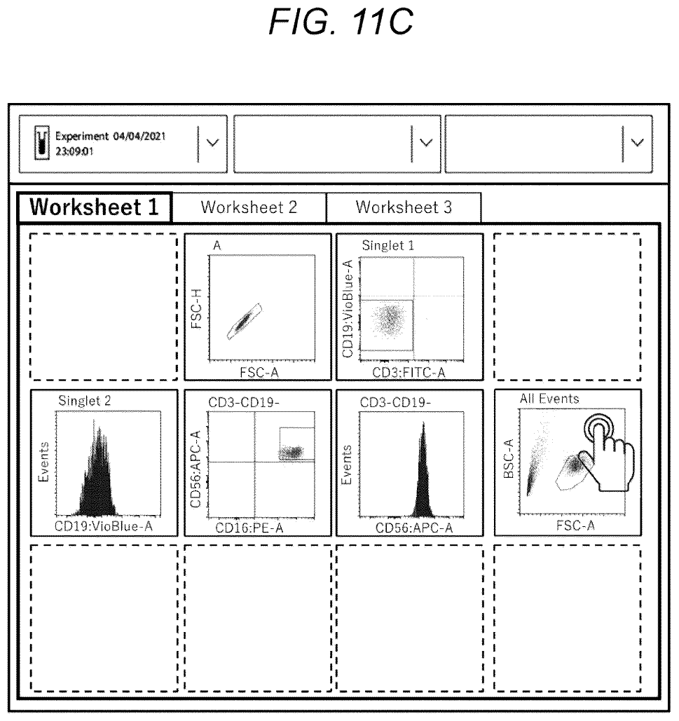

The operation of moving the measurement data to the blank spaces will be described with reference to FIGS. 11A to C. In the measurement data display area illustrated in FIGS. 11A to C, the six pieces of measurement data are displayed. Among them, as illustrated in FIG. 11A, the user touches the upper left plot data as the movement target measurement data. Next, as illustrated in FIG. 11B, the drag operation is performed to the movement destination position. In other words, the user moves a finger to the movement destination of the movement target measurement data while maintaining the state of touching the screen. When the finger reaches the movement destination position as illustrated in FIG. 11C, the user releases the finger from the screen. Therefore, the movement target measurement data is arranged at the movement destination position. An original position where the movement target measurement data exists is displayed as the blank spaces.

Such a change in the display position of the measurement data is also useful as described above with respect to the movement of the measurement data by the tap operation.

(2-2-2) Measurement Data Movement Processing to a Position where Other Measurement Data Exists
(Movement of Measurement Data to Position where Other Measurement Data Exists by Tap Operation)

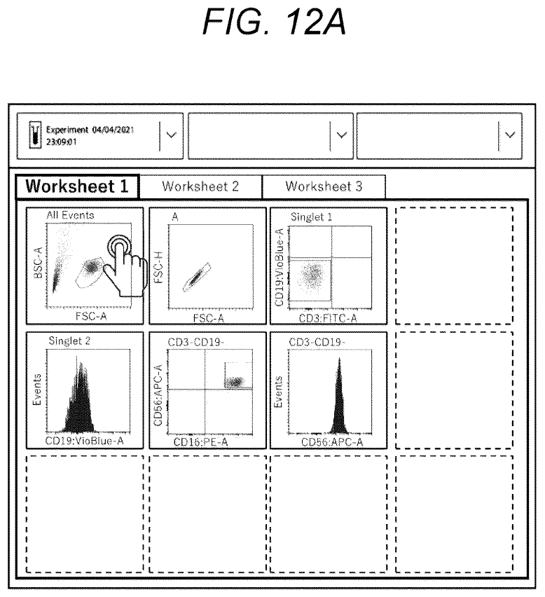

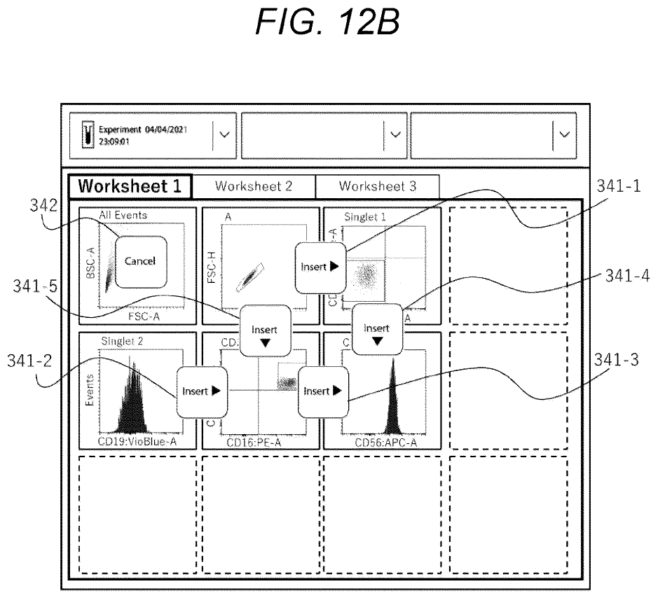

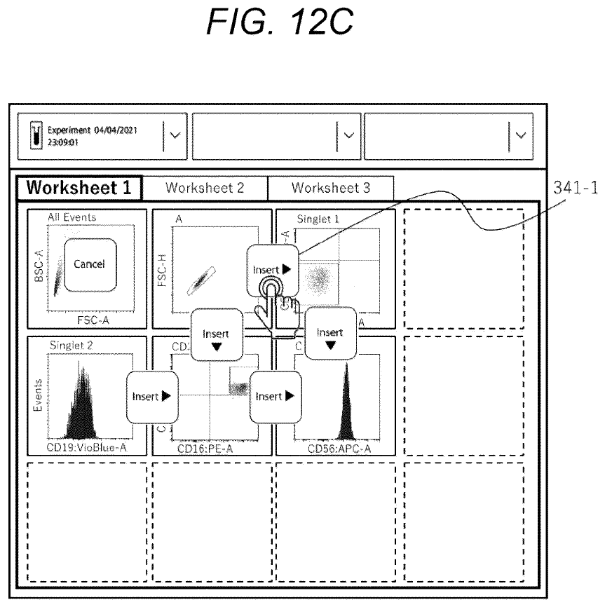

An operation of moving the measurement data to a position where other measurement data exists will be described with reference to FIGS. 12A to C. In the measurement data display area illustrated in FIGS. 12A to C, the six pieces of measurement data are displayed.

Among them, as illustrated in FIG. 12A, the user taps the upper left plot data (All Events) as the movement target measurement data. Note that in order to distinguish from the tap operation in (2-2-1) described above, the tap operation for executing the movement operation may be a different touch operation, for example, a long tap or a double tap.

In response to the tap, as illustrated in FIG. 12B, the biological particle sorting device 100 displays insertion buttons (Insert buttons) 341-1 to 341-5 that displays insertable positions of the tapped measurement data in the measurement data display area. As the measurement data is inserted, a display (a triangle in FIG. 12B) indicating a moving direction of other measurement data may be displayed on these insertion buttons.

Next, as illustrated in FIG. 12C, the user taps the insertion button 341-1, for example. In response to the tap, as illustrated in FIG. 12D, the biological particle sorting device 100 moves the measurement data (Singlet 1) existing on the right of the insertion button to the blank spaces on the right, and displays the measurement data (All Events) at the position where the measurement data (Singlet 1) existed.

Note that in a case where there is no blank spaces for movement in the row of the movement destination position, any measurement data in the row may be moved to the next row, or the insertion button may not be displayed so as not to be movable.

As described above, also in such measurement data movement processing, after the display position of the movement target measurement data is changed, the biological particle sorting device 100 may display the position where the movement target measurement data existed as blank spaces.

Such a change in the display position of the measurement data can be easily executed even by the touch operation, for example. Furthermore, since the blank spaces are used, it is possible to reduce the necessity of changing the arrangement of other measurement data before and after the change of the display position, and therefore, it is possible to prevent the user from being confused by the change of the arrangement of the measurement data. In addition, by displaying the position where the movement target measurement data exists as the blank spaces, it is easy to grasp the measurement data whose arrangement has been changed before and after the movement and the measurement data whose arrangement has been maintained before and after the movement.

In the movement process described above, as illustrated in FIG. 12B, for example, a cancel button 342 for canceling the movement of the movement target measurement data may be displayed. For example, as illustrated in FIG. 12A, in response to the user tapping the cancel button after tapping the movement target measurement data, the biological particle sorting device 100 cancels the movement processing of the movement target measurement data. In the cancel processing, the biological particle sorting device 100 deletes an insertion button group and a cancel button described above from the screen.

### (2-2-3) Processing of Deleting Rows or Columns of Blank Spaces

### (Deleting Rows or Columns of Blank Spaces by Tap Operation)

The biological particle sorting device 100 of the present disclosure may be configured to be able to delete rows or columns including only the blank spaces.

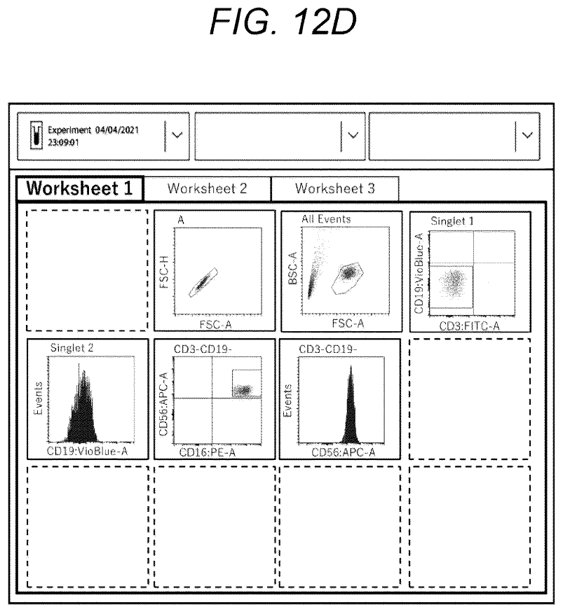

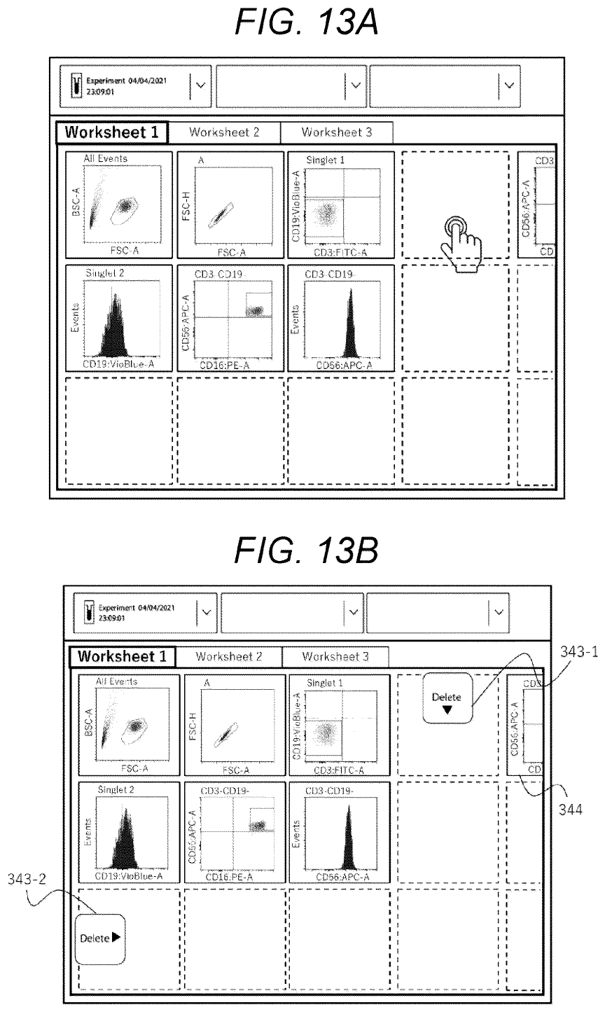

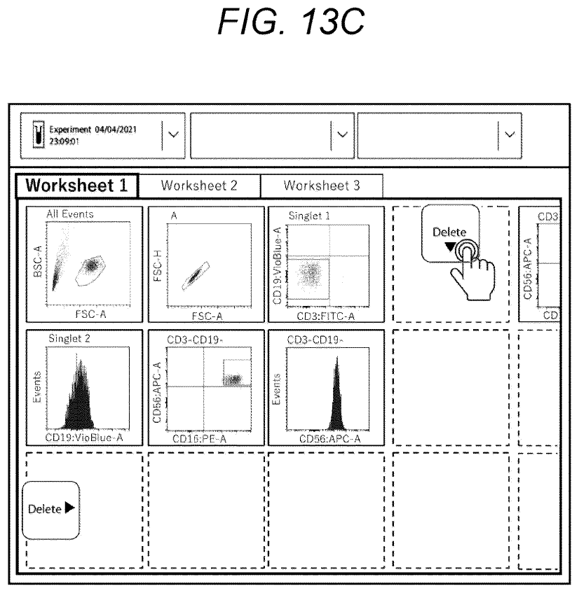

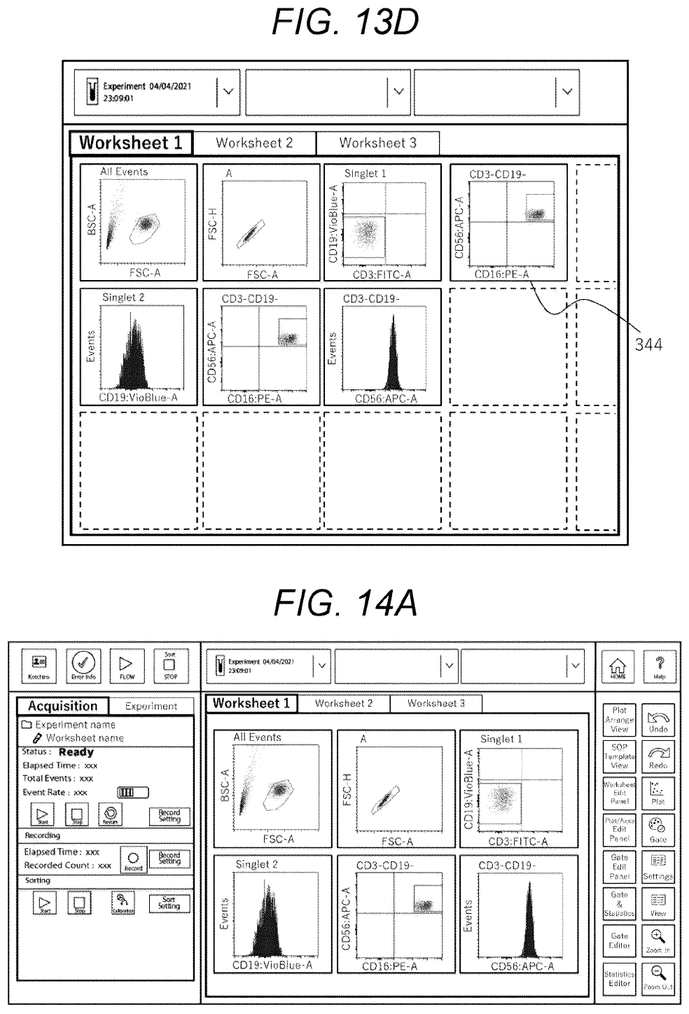

An operation of deleting rows or columns including only the blank spaces will be described with reference to FIGS. 13A to D.

In the measurement data display area illustrated in FIG. 13A, seven pieces of measurement data are displayed, and rows and columns in which only the blank spaces exist are further displayed. As illustrated in FIG. 13A, the user taps any blank space included in the rows and the columns including only the blank spaces.

In response to the tap, as illustrated in FIG. 13B, the biological particle sorting device 100 displays deletion buttons (Delete buttons) 343-1 to 343-2 for deleting the row or the column including only the blank spaces in the measurement data display area. These deletion buttons may display a display (a triangle in FIG. 13B) indicating a row or a column to be deleted.

Next, as illustrated in FIG. 13C, the user taps the delete button 343-1, for example. In response to the tap, the biological particle sorting device 100 deletes all the blank spaces in the column in which the delete button 343-1 is displayed, and moves the measurement data and the blank spaces existing on the right side of the deleted column to the left by one cell. FIG. 13D illustrates the measurement data display area after the movement. As illustrated in FIG. 13D, the measurement data 344 is moved to the position of the deleted blank spaces by the deletion.

By deleting such blank spaces, unnecessary blank spaces are deleted, and more measurement data can be displayed on the screen.

### (2-2-4) Display Method of Blank Spaces

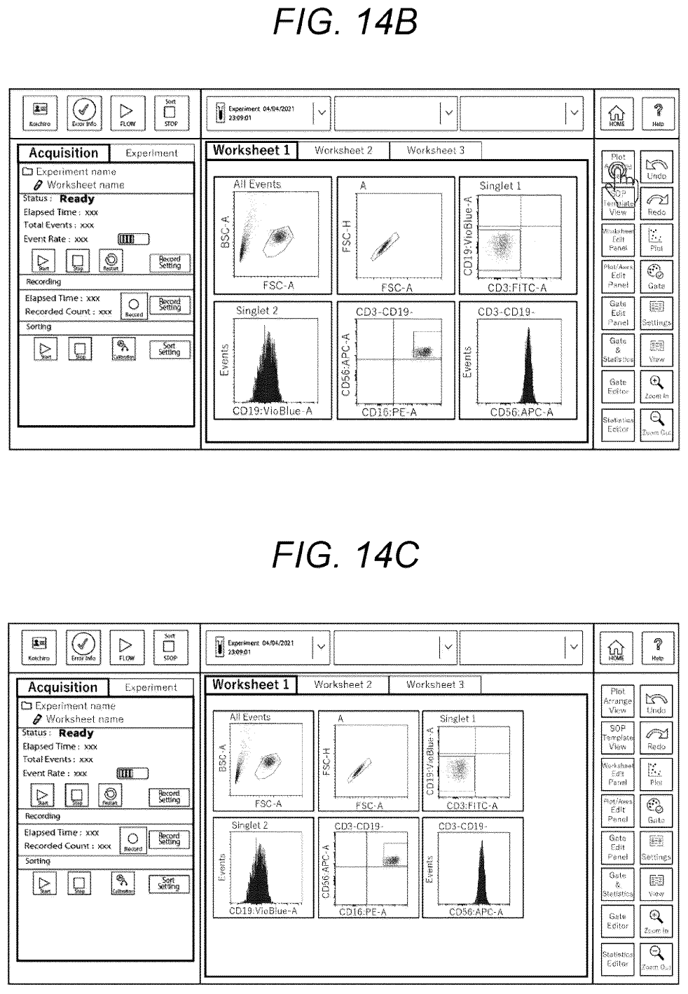

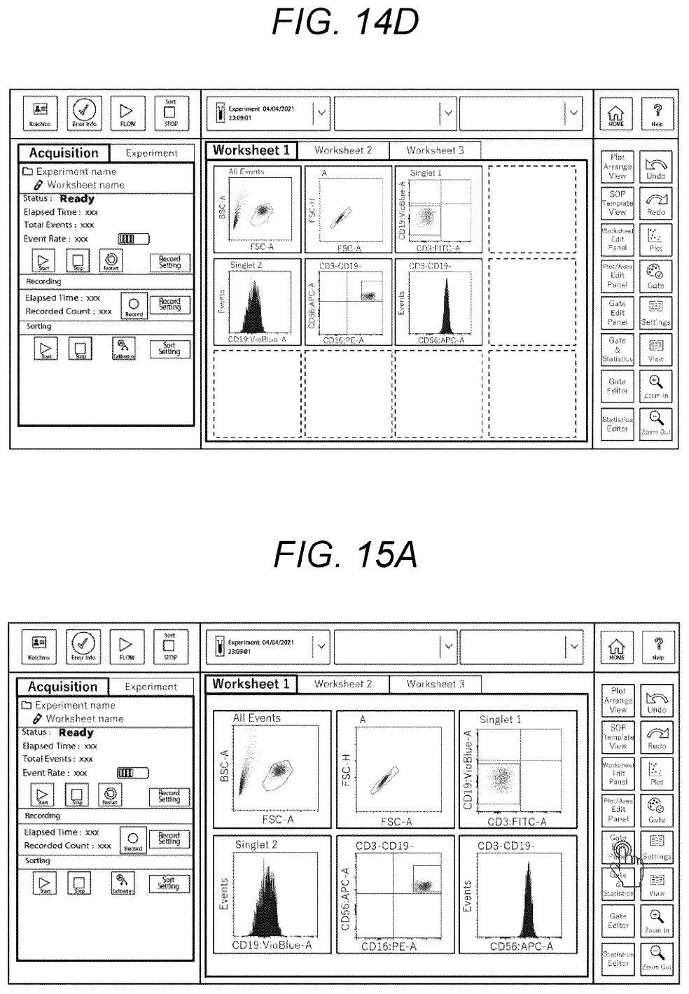

The biological particle sorting device of the present disclosure may be configured to display blank spaces in the measurement data display area in a case where a predetermined mode is selected. In a case where a mode other than the predetermined mode is selected, the blank spaces may not be displayed. The predetermined mode may be any mode in which the arrangement of the measurement data may be changed or any mode desired by the user to change the arrangement of the measurement data, and may be appropriately selected by a person skilled in the art. For example, the predetermined mode may be the Plot Arrange View mode described above. The screen transition in which the blank spaces are displayed in response to selection of the Plot Arrange View mode will be described below with reference to FIGS. 14A to D.

FIG. 14A illustrates an example of the processing condition setting screen in a state where the Plot Arrange View mode is not selected. In the measurement data display area of the screen, the six pieces of measurement data are displayed, but the blank spaces in which the measurement data can be arranged is not displayed.

It is assumed that the user who wishes to change the arrangement of the measurement data taps the Plot Arrange View button in the screen illustrated in FIG. 14A as illustrated in FIG. 14B. In response to the tapping of the button, the biological particle sorting device 100 gradually reduces the six pieces of measurement data in the measurement data display area as illustrated in FIG. 14C. The reduction is performed such that blank spaces described later are formed.

In parallel with the execution of the reduction processing of the measurement data or after the reduction is completed, the biological particle sorting device 100 displays, in the measurement data display area, the candidate position image indicating the position where the measurement data can be arranged, as illustrated in FIG. 14D. In this manner, the blank spaces appear in the measurement data display area, whereby the user can easily grasp the presence of the blank spaces.

As described above, the biological particle sorting device 100 may have a plurality of display modes, and may cause the blank spaces to appear in the measurement data display area in response to selection of the display mode that displays the blank spaces from among the plurality of display modes.

Note that the biological particle sorting device 100 may execute switching of the state of the screen from the state of the screen illustrated in FIG. 14B to the state of the screen illustrated in FIG. 14D simultaneously with the tapping without going through the process of reducing the measurement data described above with reference to FIG. 14C.

Furthermore, the blank spaces may not be displayed only by tapping the Plot Arrange View button. In this case, after tapping the Plot Arrange View button, the biological particle sorting device 100 may moreover, display the blank spaces as described above in response to the selection (for example, tapping or dragging) of any movement target measurement data.

The blank spaces are caused to appear in the measurement data display area in response to selection of a display mode that displays the blank spaces from among the plurality of display modes and selection of movement target measurement data. As described above, the biological particle sorting device 100 may have the plurality of display modes, and may cause the blank spaces to appear in the measurement data display area in response to the selection of the display mode that displays the blank spaces from among the plurality of display modes and selection of the movement target measurement data.

### (2-3) Operation Panel

The biological particle sorting device 100 of the present disclosure may be configured to be able to display various operation panels for adjusting the measurement data or adjusting the sorting processing conditions (particularly, gate setting) for the measurement data. It may be displayed in the processing condition setting screen in response to tapping a predetermined operation panel call button, and in particular, it may be displayed in the measurement data display area. By displaying various operation panels in a case where it is necessary, it is possible to reduce an adverse effect on visibility of the measurement data. In one preferred embodiment, these operation panels may be translucent. More specifically, the operation panel may be displayed in the measurement data display area, and may be displayed translucently so that the measurement data in the measurement data display area can be visually recognized. Therefore, it is possible to execute an operation using the operation panel while confirming the measurement data.

An example of the operation panel will be described below.

### (2-3-1) Gate Edit Panel

The biological particle sorting device 100 may be configured to be able to display the gate edit panel that edits the gate set in the measurement data.

For example, as illustrated in FIG. 15A, the user taps a gate edit panel button (Gate Edit Panel button) on a processing condition setting screen 300. In response to the tap, as illustrated in FIG. 15B, the biological particle sorting device 100 displays a gate edit panel 351 in the screen, particularly in the measurement data display area. As shown in FIG. 15B, the gate edit panel may be opaque, that is, displayed so as to hide the measurement data behind it. Alternatively, the gate edit panel may be translucent or transparent, that is, the measurement data behind the gate edit panel may be displayed so as to be visible through the gate edit panel. For example, as shown in FIG. 15C, a translucent gate edit panel 352 may be displayed in the measurement data area. Since the panel is translucent, gate editing can be performed while the measurement data behind the panel is visually recognized.

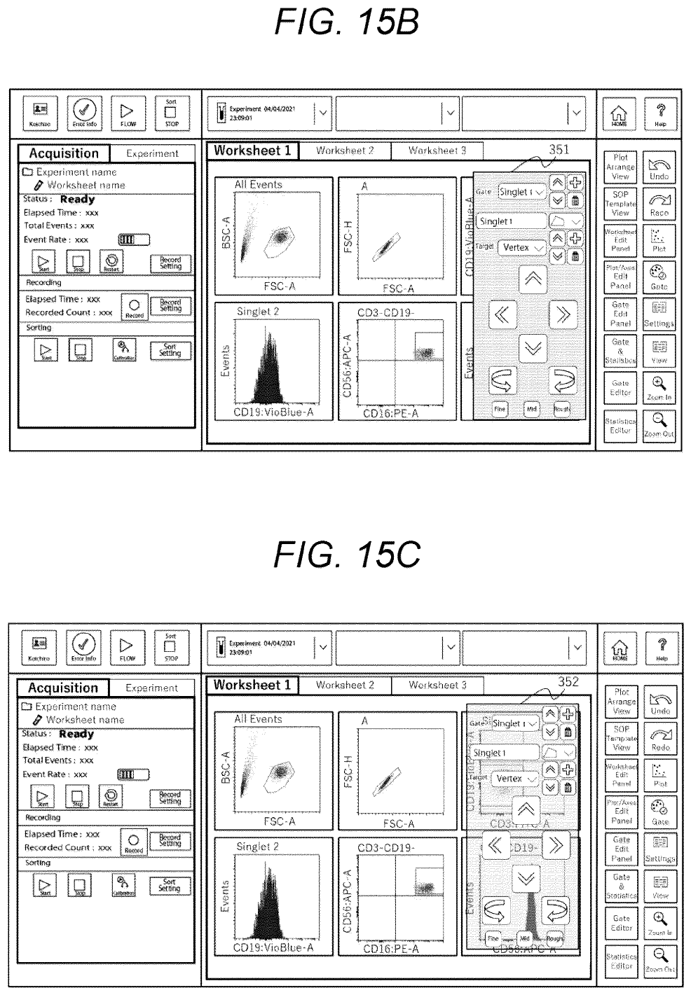

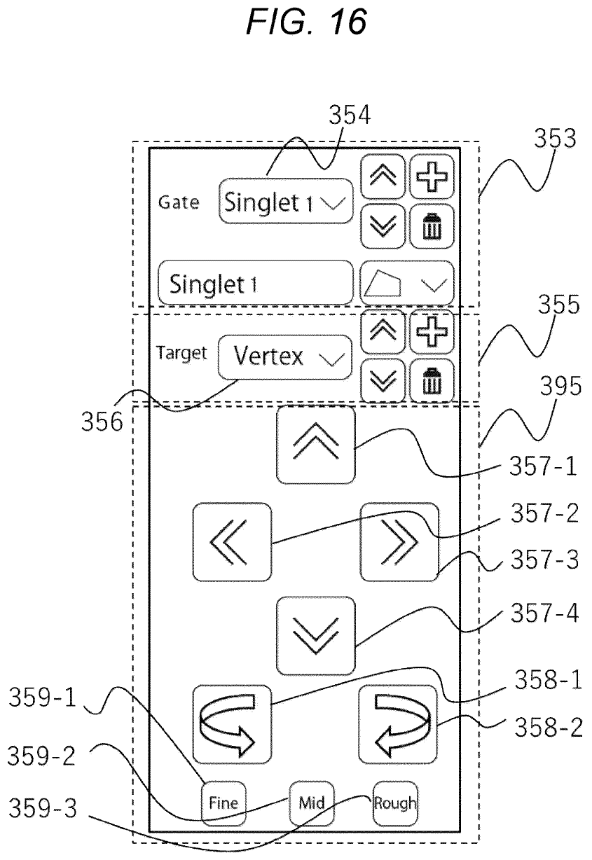

The gate edit panel is an operation panel that edits the gate set in the measurement data. The gate edit panel will be described below with reference to FIG. 16.

The gate edit panel illustrated in FIG. 16 includes a gate selection area 353 that selects the gate to be edited. The gate selection area includes, for example, a drop-down menu 354 that selects the gate to be edited. In response to selecting the drop-down menu, the biological particle sorting device 100 displays a list of editable gates. The user can select a gate desired to be edited from the list. Furthermore, the another operation button that selects the gate to be edited or the name of the selected gate may be displayed in the area. In FIG. 16, “Singlet 1” is displayed below the drop-down menu as the selected gate name. As described above, the gate edit panel may be configured to be able to select the gate to be edited.

The gate edit panel moreover, includes an operation target selection area 355 that selects a configuration component to be operated among the gate configuration components selected in the gate selection area. The operation target selection area includes a drop-down menu 356 that selects an operation target. The drop-down menu lists edit target items of the gate selected in the drop-down menu 354 in the gate selection area 353.

In other words, the biological particle sorting device 100 changes the selectable configuration components in the operation target selection area according to the gate selected in the gate selection area. The biological particle sorting device 100 acquires edit target item data of a gate in response to selection of the gate in the gate selection area. Then, the biological particle sorting device 100 generates the drop-down menu displayed in the operation target selection area based on the edit target item data.

In a case where the gate is a quadrangle, the edit target items listed may include, for example, the entire gate and four vertices constituting the gate.

For example, in a case where the gate is a polygon other than a quadrangle, the edit target items listed can include the entire gate, the four vertices of the quadrangle set to surround the entire gate, and a vertex group of the polygon.

The edit target items listed may include, for example, in a case where the gate is circular or elliptical, the entire gate, the four vertices of the quadrangle set to surround the entire gate, and a center of the circular or elliptical shape.

As described above, the gate edit panel may be configured to be able to select the entire selected gate or the configuration component of the selected gate as the object to be edited.

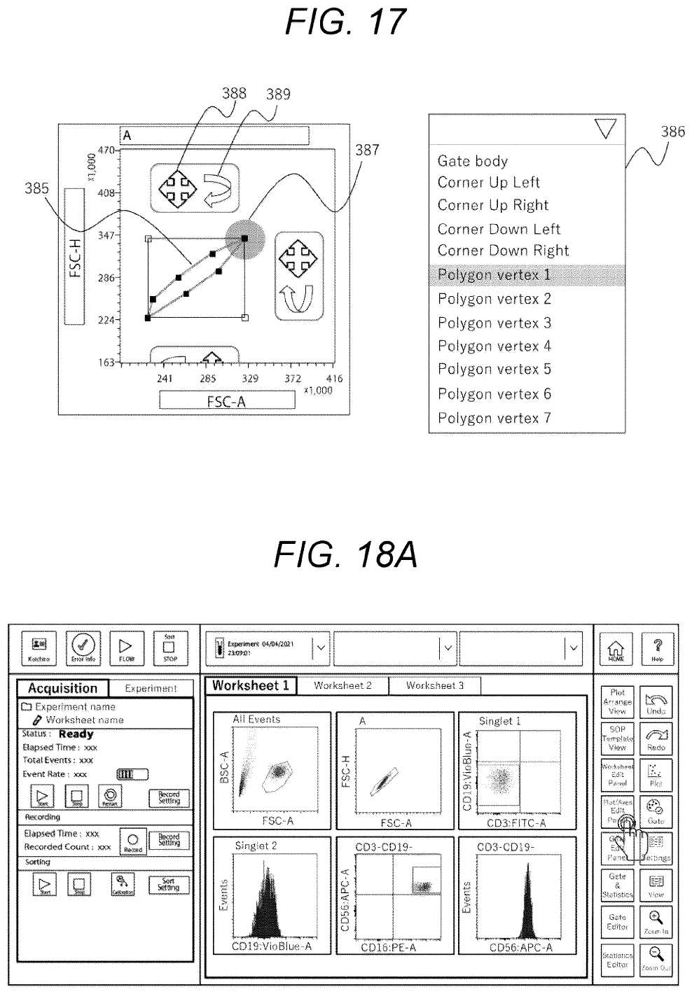

An example of the drop-down menu will be described below with reference to FIG. 17. In FIG. 17, an example of a gate selected in the operation target selection area of the gate edit panel (left in FIG. 17) and an example of the drop-down menu in which the edit target items of the gate are listed (right in FIG. 17) are displayed. A gate 385 illustrated on the left of FIG. 17 is a polygonal gate. The edit target items of the gate include the entire gate body, the four vertices of the quadrangle (Corner Up left, Corner Up Right, Corner Down Left, and Corner Down Right) set to surround the entire gate, and the vertices of the polygon (Polygon vertex 1-7). These edit target items are listed in a drop-down menu 386 illustrated on the right of FIG. 17. In the drop-down menu, Polygon vertex 1, which is one of the vertices of the polygon, is selected. A mark 387 is attached to the edit target items selected in the drop-down menu on the left of FIG. 17, so that the user can confirm the selected edit target item.

In the preferred embodiment, a movement handle button 388 for moving the gate or the configuration component thereof and/or a rotation handle button 389 for rotating the gate are displayed around the gate selected as the operation target. As illustrated in FIG. 17, a plurality of these buttons may be displayed so as to surround the selected gate, and for example, a total of four buttons may be displayed vertically and horizontally. In response to the user tapping the movement handle button, the biological particle sorting device 100 moves the gate or the configuration component thereof. Furthermore, in response to the user tapping the rotation handle button, the biological particle sorting device 100 rotates the gate or the configuration component thereof.

Furthermore, the movement operation or the rotation operation of the gate or the configuration component thereof selected as the operation target may be executed by a button displayed in an operation button area 395 in the gate edit panel. In the same area, movement handle buttons 357-1 to 357-4 for performing the movement operation and rotation handle buttons 358-1 to 358-2 for performing the rotation operation are shown. The biological particle sorting device 100 moves or rotates the gate or the configuration component thereof in response to the tapping of these buttons.

The area 395 may moreover, include movement amount adjustment buttons 359-1 to 359-3 for adjusting movement amount by tapping the movement handle button or the rotation handle button described above. The biological particle sorting device 100 changes the movement amount in response to tapping of these buttons. In the area 395, three buttons of “Fine”, “Mid”, and “Rough” are displayed, that is, the movement amount can be adjusted in three stages, but the number of adjustment stages is not limited thereto. Furthermore, the biological particle sorting device 100 may display a button for increasing or decreasing the movement amount.

### (2-3-2) Plot/Axis Edit Panel

The biological particle sorting device 100 may be configured to be able to display the plot or axis edit panel (also referred to as a “plot/axis edit panel button”) that edits a display form and/or axis of the measurement data.

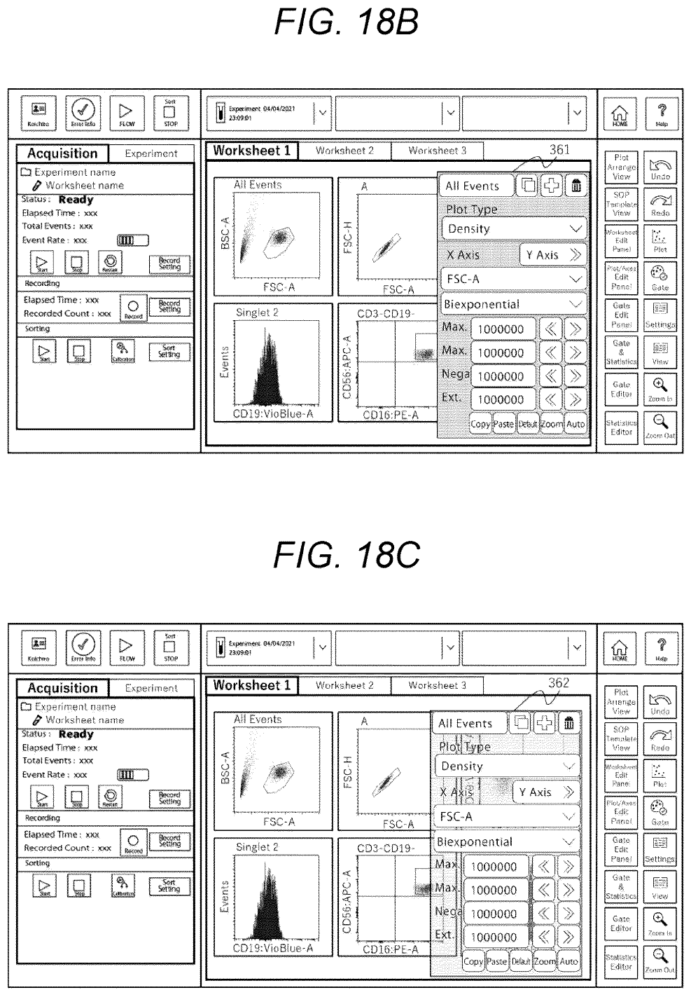

For example, as illustrated in FIG. 18A, the user taps the plot/axis edit panel button (Plot/Axes Edit Panel button) on the processing condition setting screen 300. In response to the tap, the biological particle sorting device 100 displays a plot/axis edit panel 361 on the screen, in particular, in the measurement data display area, as illustrated in FIG. 18B. As shown in FIG. 18B, the plot/axis edit panel may be opaque, that is, displayed so as to hide the measurement data behind it. Alternatively, the plot/axis edit panel may be translucent or transparent, that is, the measurement data behind it may be displayed so as to be visible through the plot/axis edit panel. For example, as shown in FIG. 18C, a translucent plot/axis edit panel 362 may be displayed within the measurement data area. Since the panel is translucent, it is possible to edit the measurement data or the axis thereof while visually recognizing the measurement data behind the panel.

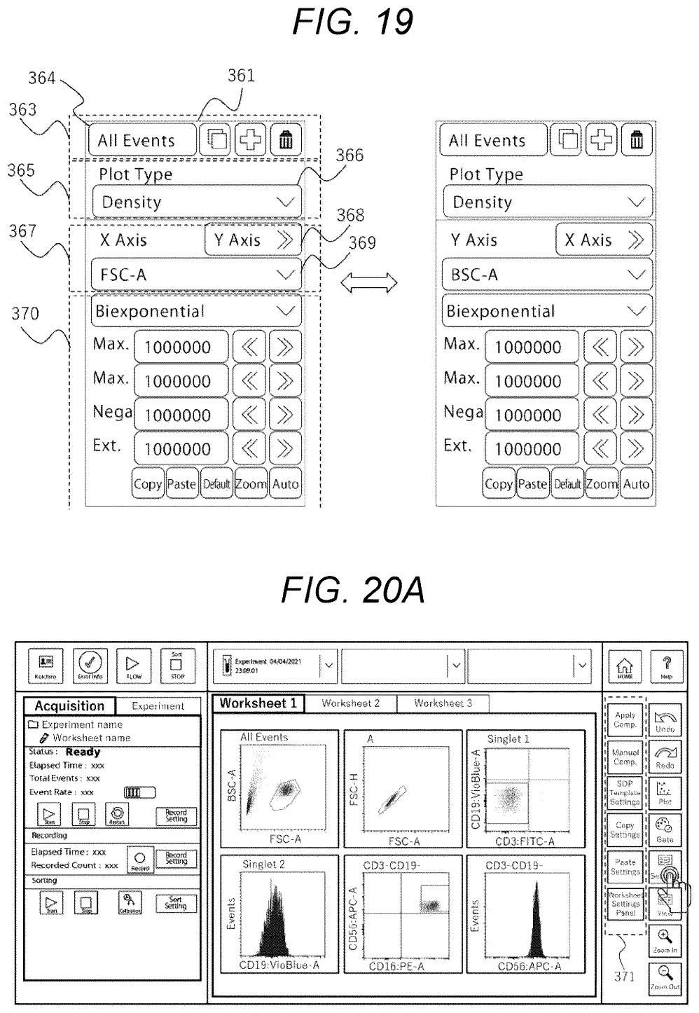

The plot/axis edit panel is an operation panel that edits measurement data and/or the axes thereof. The plot/axis edit panel will be described below with reference to FIG. 19.

The plot/axis edit panel 361 shown on the left of FIG. 19 includes a measurement data selection area 363 that selects measurement data to be edited. The measurement data selection area may include, for example, a drop-down menu 364 that selects the measurement data to be edited. Furthermore, another operation button that selects the measurement data to be edited may be displayed. The name of the selected measurement data may be displayed in the area.

The plot/axis edit panel moreover, includes a plot type selection area 365 that selects a type of display form of the measurement data (for example, a type of plot). The plot type selection area includes, for example, a drop-down menu 366 that selects a plot type of the measurement data to be edited. For example, one or more of a density plot, a dot plot, a histogram, and a contour plot may be listed in the drop-down menu. In response to the user selecting the plot type from the drop-down menu, the biological particle sorting device 100 changes the plot type of the measurement data to be edited to the selected type of plot.

The plot/axis edit panel moreover, includes an axis edit area 367 that edits the axis. The axis edit area includes an axis selection button 368 that selects or changing an axis to be operated. For example, in the left of FIG. 19, the axis to be operated is an X axis, but in response to the user tapping the axis selection button 368 to select a Y axis, the biological particle sorting device 100 changes the operation target to the Y axis as illustrated in the right of FIG. 19. As described above, the plot or axis edit panel may be configured to allow selection of the axis to be edited.

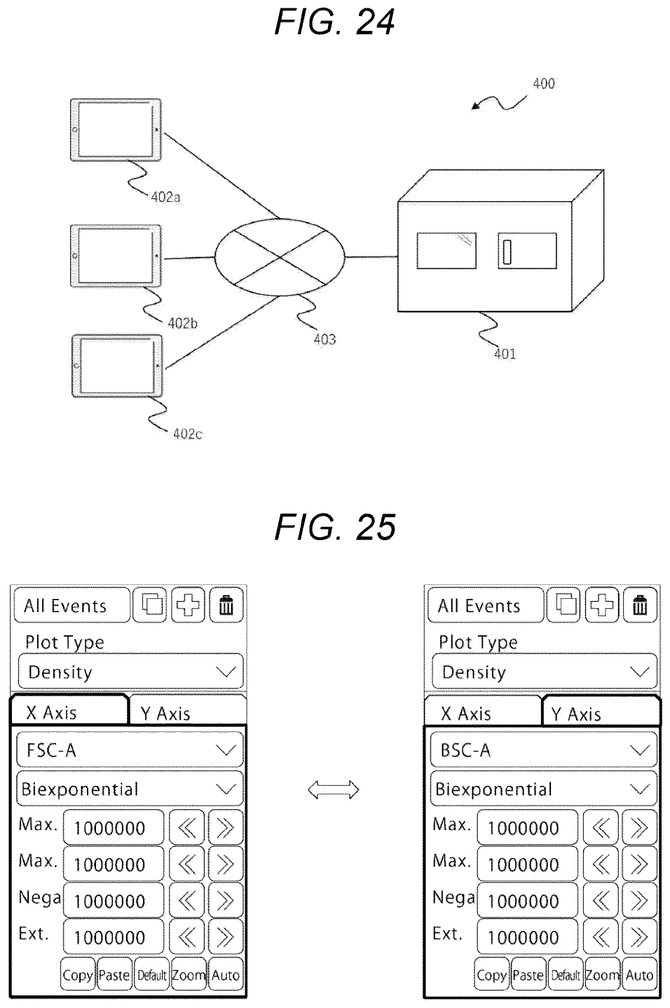

Furthermore, the method of changing the axis to be operated is not limited to the changing method using the button illustrated in FIG. 19, and may be, for example, a changing method using a tab. The change by this tab will be described with reference to FIG. 25. In the left of FIG. 25, an X-axis tab is selected, that is, the X-axis tab is active. In the left, the X-axis tab being active is expressed by the X-axis tab surrounded by a thick line and an inactive Y-axis tab (the Y axis) surrounded by a thin line, but an expression method is not limited thereto. As the Y-axis tab in the left is tapped, the display is changed as illustrated in the right of FIG. 25. In the right, the Y-axis tab is active. In the right, activation of the Y-axis tab is similarly expressed by the Y-axis tab surrounded by the thick line and the inactive X-axis tab surrounded by the thin line. Furthermore, contents of the axis at a lower portion is also changed according to the selection of the tab. In FIG. 25, in a case where the X-axis tab is active, it is shown that the X-axis is for forward scattered light (FSC-A) (left), and in a case where the Y-axis tab is active, it is shown that the Y-axis is for backward scattered light (BSC-A) (right).

Furthermore, the axis edit area may include a drop-down menu 369 that selects a type of light or fluorescent dye to be adopted as the axis. Examples of the type of the light can include scattered light (the forward scattered light, the backward scattered light, or the side scattered light). In a case of the fluorescent dye, a target molecule to be captured of the antibody labeled with the fluorescent dye may be displayed.

Furthermore, the axis edit area includes a setting area 370 for setting an axis format and/or a numerical range. The area may be configured such that a type of axis (for example, a logarithmic axis or a linear axis) and/or a numerical range of the axis (for example, a maximum value and/or a minimum value of the axis) can be set. The axis edit area may moreover, include various operation buttons. For example, as illustrated in FIG. 19, a copy button (Copy button) for copying a plot or an axis type and/or a numerical range setting, a paste button (Paste button) for pasting a plot or an axis type and/or a numerical range setting, a default button (Default button) for returning a plot (or an axis) setting to a default, a zoom button (Zoom button) for zooming a plot, an automatic setting button (Auto) button for automatically setting a plot (or an axis), and the like may be displayed. All of these buttons may be displayed, or any one or more of these buttons may be displayed in the area.

### (2-3-3) Measurement Data Display Area Setting Panel

The body particle sorting device 100 may be configured to be able to display a measurement data display area setting panel (also referred to as a “worksheet setting panel” in the present description) for adjusting the display setting of the measurement data display area (particularly, the worksheet).

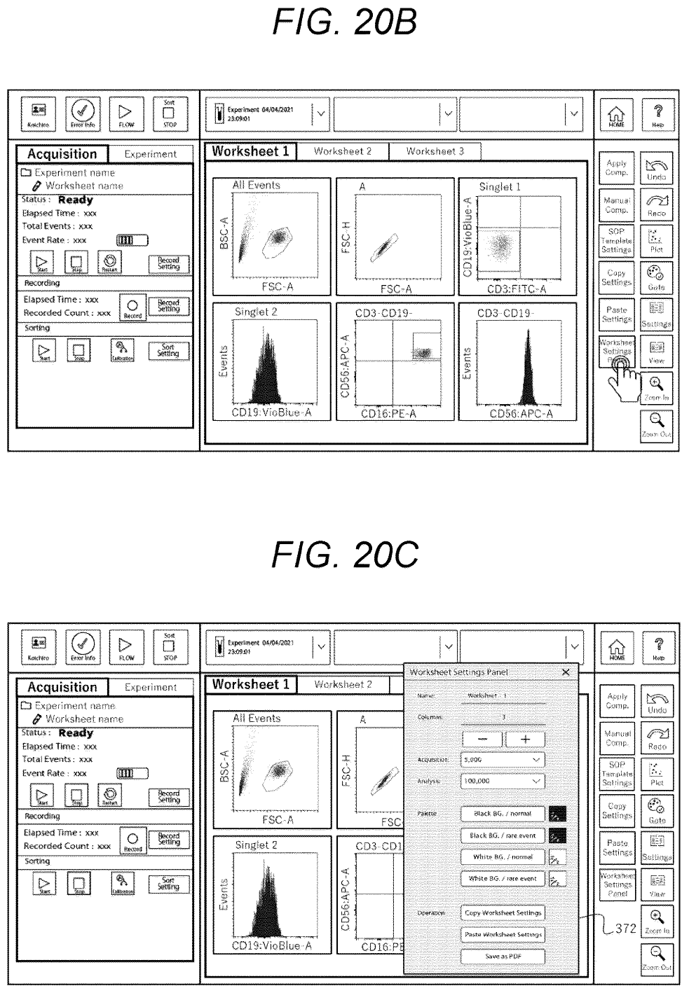

For example, as illustrated in FIG. 20A, in response to the user's tapping of the setting button (Settings button) on the processing condition setting screen 300, the biological particle sorting device 100 displays a button group 371 related to various settings in the third display area. Next, as illustrated in FIG. 20B, the user taps a worksheet setting button (Worksheet Settings button) in the button group. In response to the tap, as illustrated in FIG. 20C, the biological particle sorting device 100 displays a worksheet setting panel 372 in the screen, particularly in the measurement data display area. As shown in FIG. 20C, the worksheet setting panel may be opaque, that is, may be displayed so as to hide the measurement data behind it. Alternatively, the worksheet setting panel may be translucent or transparent, that is, the measurement data behind it may be displayed so as to be visible through the worksheet setting panel. For example, as illustrated in FIG. 20D, a translucent worksheet setting panel 373 may be displayed in the measurement data area. Since the panel is translucent, it is possible to adjust the display setting of the worksheet while visually recognizing the measurement data behind the panel.

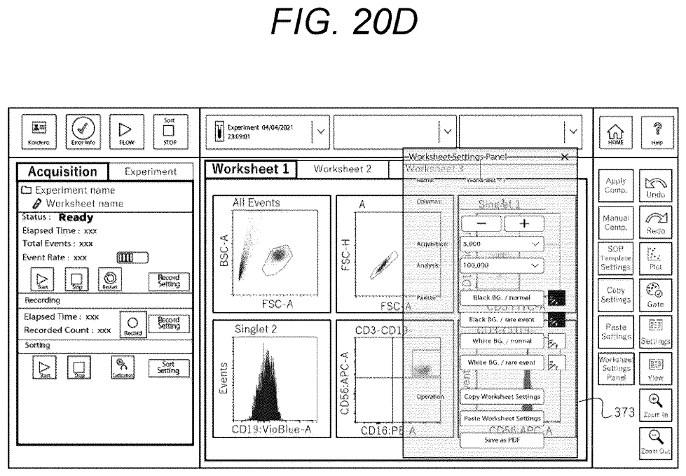

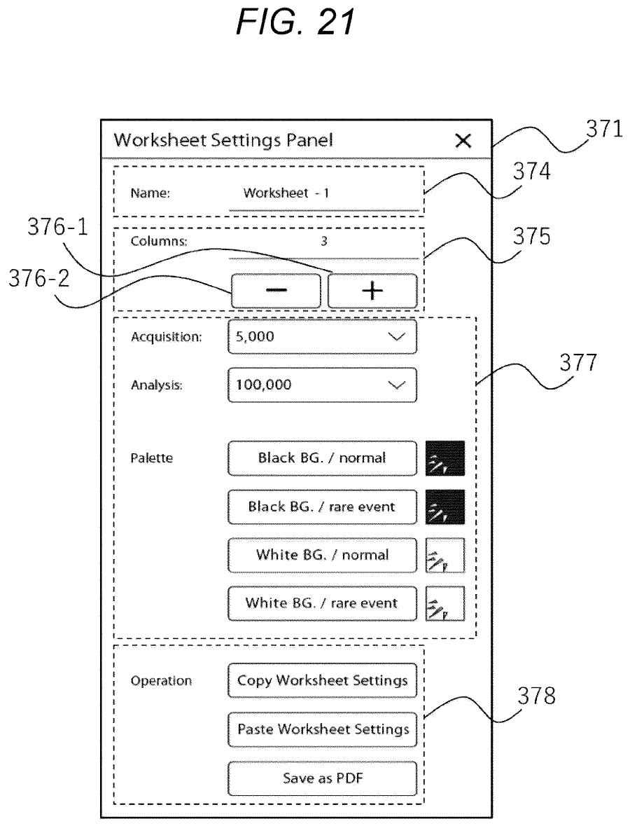

The worksheet setting panel will be described below with reference to FIG. 21.

The worksheet setting panel 371 illustrated in FIG. 21 includes an area 374 that displays a worksheet name to be set. In this area, for example, the worksheet name of the active tab may be displayed.

Furthermore, as illustrated in FIG. 21, the panel may moreover, include a number-of-columns setting area 375 for setting the number of columns of the measurement data displayed in the area. In FIG. 21, Columns is displayed in the area, and 3 is set as the number of columns. In other words, it is set that three pieces of measurement data are displayed side by side per row in the measurement data display area. In a case where it is set in this way, for example, as illustrated in FIG. 20A, the biological particle sorting device 100 displays the measurement data in three columns in the measurement data display area. Then, in FIG. 20A, the six pieces of measurement data are displayed in an arrangement configuration of 3 columns×2 rows, but as the number of pieces of measurement data increases, the biological particle sorting device 100 increases the number of rows while fixing the number of columns.

The column of the blank spaces in (2-2) described above may not be counted as the number of columns displayed in the same area. In other words, in a case where the mode that displays the blank spaces in (2-2) described above is selected by the user, in such a mode, the biological particle sorting device 100 can arrange, in the measurement data display area, the measurement data of the number of columns obtained by adding the number of columns of the blank spaces to the number of columns set in the number-of-columns setting area 375. For example, in a case where 3 is selected as the number of columns in the number-of-columns setting area 375 as illustrated in FIG. 20A and the blank spaces of one column are displayed in the mode, four columns of measurement data can be arranged in the measurement data display area in the mode.

As described above, the biological particle sorting device 100 may be configured to increase the number of columns at which the measurement data can be arranged in the measurement data display area in response to the selection of the display mode that displays the blank spaces. Then, the increased columns may be displayed as the blank spaces.

The number-of-columns setting area 375 may include a button for increasing or decreasing the number of columns of one or more pieces of measurement data displayed in the measurement data display area. In FIG. 21, a button 376-1 for increasing the number of columns and a button 376-2 for decreasing the number of columns are displayed, but a display method of these buttons may be appropriately selected by a person skilled in the art. In response to the user tapping these buttons, the biological particle sorting device 100 increases or decreases the number of columns of measurement data arranged in the measurement data display area (and the number of columns displayed in the area 375).

As described above, the biological particle sorting device according to the present disclosure may be configured such that the number of columns at which the measurement data can be arranged in the measurement data display area can be set. For example, the biological particle sorting device may be configured to display a setting panel that changes the number of columns.

It is desirable that the manner of arranging the measurement data used for the biological particle sorting can be appropriately changed according to, for example, a type of an object to be sorted, a manner of setting the gate, or a desire of the user. With such a configuration, the biological particle sorting device according to the present disclosure can adjust arrangement of one or more pieces of measurement data as desired.

Furthermore, as illustrated in FIG. 21, the panel may further include an area 377 for adjusting the number of events in the measurement data displayed on the worksheet and/or the color of the measurement data and/or an operation area 378 for copying, pasting, or saving the worksheet settings.

### (2-4) Display Processing of Superimposing and Displaying Contents of Other Display Areas on Measurement Data Display Area

The biological particle sorting device according to the present disclosure, as described above, displays the first display area (the sorting operation control area) and/or the third display area (the operation button area) in addition to the second display area (the measurement data display area). Here, the number of pieces of measurement data displayed in the second display area is one or more, and is plural in many cases. It is desirable that the measurement data is displayed in a larger size, but it is desirable that the first display area and/or the third display area is also appropriately displayed for setting processing conditions and the sorting operation.

Therefore, in the preferred embodiment, the biological particle sorting device according to the present disclosure is configured to be able to display the display contents of the first display area and/or the third display area in superposition with the second display area in response to a user operation. For example, the biological particle sorting device may be configured to display the display contents of the first display area or the third display area to be superimposed on the second display area in a case where the user executes a predetermined screen operation, and not to display the display contents in other cases. An example of such display processing will be described below.

(2-4-1) Embodiment in which Display Contents in First Display Area are Displayed in Superposition with Second Display Area

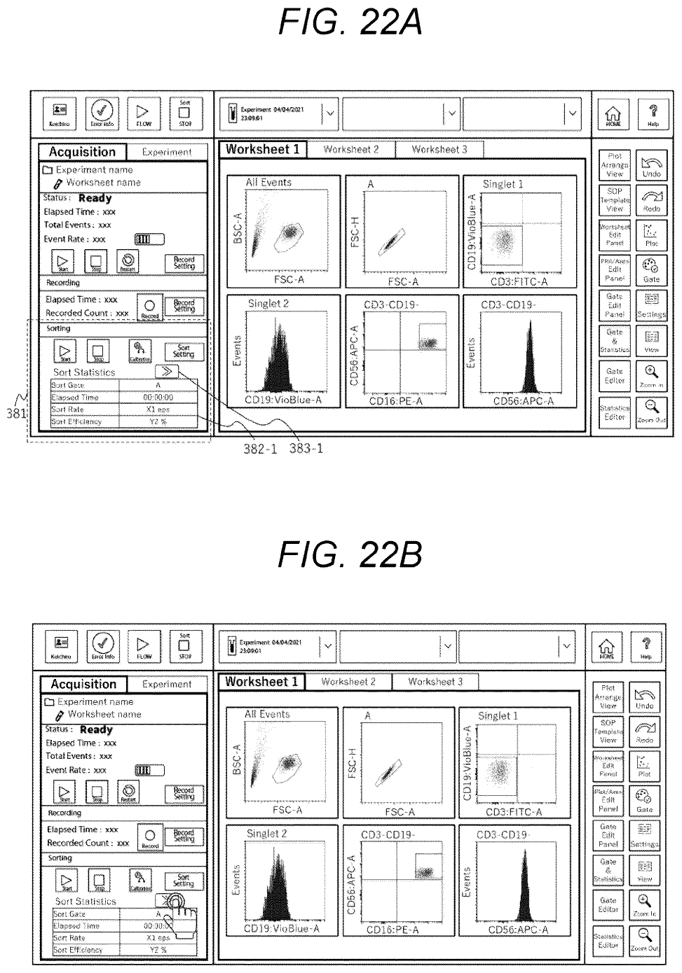

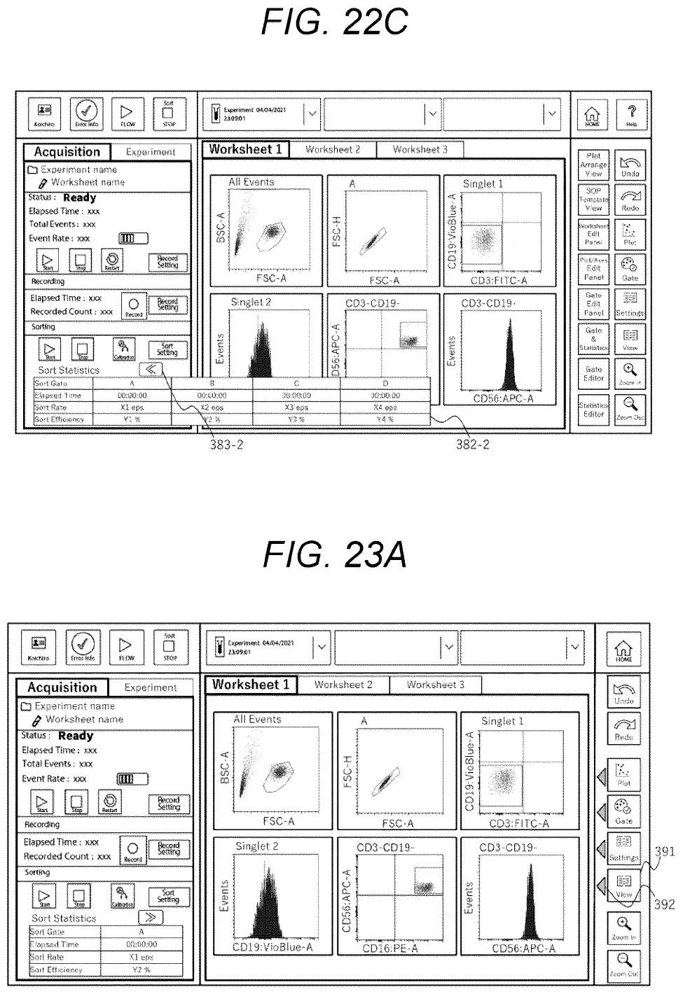

As illustrated in FIG. 22A, various numerical data or measurement data acquired by the biological particle sorting device 100 may be displayed in the first display area. These data may be displayed in a tabular form, as shown in a table 382-1, in an area 381 including one or more buttons that controls the sorting operation.

The table 382-1 shows data acquired by the biological particle sorting device 100 in sorting by a gate A. In the table 382-1, a name of the gate, a period of time (Elapsed Time) elapsed from a start of sorting by the gate, a sorting rate (Sor Rate) of the gate, and a sorting efficiency (Sort Efficiency) of the gate are displayed, but the data displayed in the table is not limited thereto. For example, the remaining period of time (Remaining Time) during which the sorting is performed by the gate, the number of times (Sort Count) of executing the sorting operation by the gate, the number of abort times (Abort Count) at the gate, and the like may be displayed.

For example, the sorting process may be performed by a plurality of gates different from each other, and data regarding the sorting process may be acquired for each gate. A table that displays these pieces of data cannot be stored in the first display area, or when the table is stored in the first display area, characters indicating the data in the table become extremely small, and it becomes difficult for the user to confirm the data.

Therefore, the biological particle sorting device 100 may be configured to be able to expand and display the display contents of the first display area on the second display area. The biological particle sorting device 100 may display a data expansion button for performing such display. For example, as illustrated in FIG. 22A, the biological particle sorting device 100 can display a data expansion button 383-1 in the first display area.

As shown in FIG. 22B, the user taps the data expansion button 383-1. In response to the tap, the biological particle sorting device 100 displays a table 382-2 as illustrated in FIG. 22C. A horizontal length of the table 382-2 is larger than a width of the first display area, and is displayed so as to overlap with the second display area. The table 382-2 displays data acquired for gates B-D as well as data acquired for gate A. As described above, by superimposing and displaying the table 382-2 on the second display area, a character size in the table can be increased, and the user can easily confirm the table data.

Furthermore, in FIG. 22C, a data folding button 383-2 is displayed instead of the data expansion button 383-1. In response to the user tapping the data folding button 383-2, the biological particle sorting device 100 folds the table data back to a state of FIG. 22A.

As described above, the biological particle sorting device 100 may be configured to be able to expand and display the data associated with the items in the first display area on the second display area. Furthermore, the biological particle sorting device 100 may be configured to be able to fold the expanded data. With such an expanding process and a folding process, the user can check the data only when necessary, and can maintain the visibility of the measurement data in the second display area. In addition, on a screen having a limited size, data can be checked only in a case where necessary.

(2-4-2) Embodiment in which Display Contents in Third Display Area are Displayed in Superposition with Second Display Area

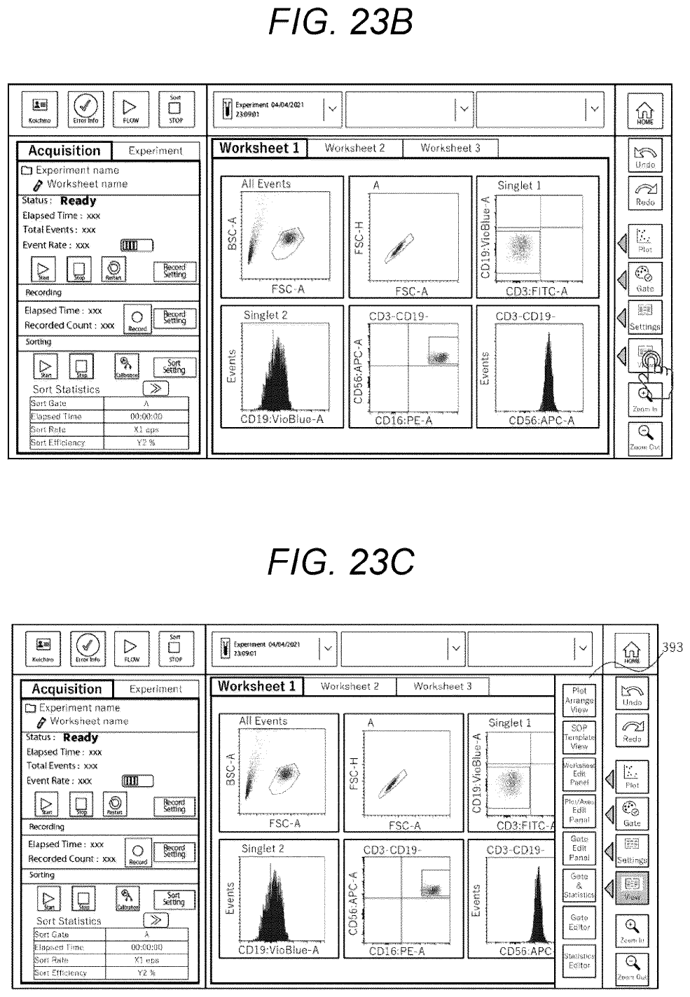

In the third display area, as illustrated in FIG. 23A, various operation buttons used for setting the sorting processing conditions are displayed. By reducing the area in which these operation buttons are displayed, the second display area can be made larger, and the measurement data can be more easily viewed. Therefore, the biological particle sorting device 100 may be configured to be able to expand and display the display contents of the third display area on the second display area. The biological particle sorting device 100 may display an operation button capable of such expansion processing in the third display area. For example, as illustrated in FIG. 23A, the biological particle sorting device 100 can display an expansion button 391 (View button) in the third display area. A display 392 indicating that the button is expandable may be attached to a side of the expansion button 391. Note that in FIG. 23A, the Plot button, the Gate button, and the Settings button with the same display can also be expanded.

As shown in FIG. 23B, the user taps the expansion button 391. In response to the tap, the biological particle sorting device 100 displays an area 393 in which an operation button group is displayed as illustrated in FIG. 23C. The area 393 is displayed so as to overlap with the second display area.

In response to the user tapping the expansion button 391 again, the area 393 may be collapsed.

As described above, the biological particle sorting device 100 may be configured to be able to expand an area, which includes one or more operation buttons associated with the operation buttons in the third display area, on the second display area. Furthermore, the biological particle sorting device 100 may be configured to be able to fold the expanded area. By such an expanding process and a folding process, the operation button group is displayed only when the user needs it, and the visibility of the measurement data in the second display area can be maintained. In addition, the operation button group can be confirmed only in a case where necessary on a screen having a limited size.

### (3) Modification Example

In the (1) and (2) described above, the biological particle sorting device that executes the sorting processing on the biological particles contained in the biological sample flowing through a flow channel (particularly, the biological particles flowing side by side in the flow channel) has been described. However, the present disclosure may be applied to a biological particle sorting device that executes the sorting processing on a biological particle group existing two-dimensionally or three-dimensionally. In other words, the biological particle sorting device of the present disclosure may be configured to execute the sorting processing on the biological particle group existing two-dimensionally or three-dimensionally. Examples of the biological particle sorting device configured as described above include a biological particle sorting device that executes the sorting processing on a group of biological particles existing in a well arranged two-dimensionally or three-dimensionally, a biological particle sorting device that executes the sorting processing on a group of biological particles existing two-dimensionally or three-dimensionally on an arbitrary support (for example, a cell culture surface or a cell immobilization surface), and a biological particle sorting device that executes the biological particle sorting processing on a group of biological particles (for example, a biological tissue) forming a three-dimensional structure.

Examples of the biological particle sorting device that executes sorting processing on the group of biological particles existing in the well include a biological particle sorting device that executes biological particle sorting processing using a particle capturing chip described in JP 2020-174598 A.

Examples of the biological particle sorting device that executes sorting processing on the group of biological particles existing two-dimensionally on the arbitrary surface include a biological particle sorting device that sorts a specific biological particle from a group of biological particles (cells) immobilized via a linker (for example, a photodegradable linker) decomposable on a certain surface can be mentioned.

These devices may be configured to acquire a fluorescence signal or a fluorescence image of the biological particle using, for example, an optical detector including a microscope or the like, and identify the biological particle to be sorted based on the acquired fluorescence signal or fluorescence image.

These devices may be configured to remove only the biological particle identified in this manner from the well, or may be configured to release the immobilization of only the biological particle identified in this manner (for example, to cleave a linker that immobilizes the biological particle).

These devices may be configured to execute the display processing in (1) and (2) described above.

### 3. Second Embodiment of Present Disclosure (Biological Particle Sorting System)

The present disclosure also provides the biological particle sorting system including the display unit that displays a measurement data display area in which the one or more pieces of measurement data are displayed. The system may be configured to display the blank spaces in addition to the one or more pieces of measurement data in the measurement data display area, display the movement target measurement data at the movement destination position in response to the user selecting one movement target measurement data of the one or more pieces of measurement data and one movement destination position in the blank spaces, and display a position where the movement target measurement data existed as the blank spaces after the display position of the movement target measurement data is changed. Such display processing may be executed by the biological particle sorting device or the information processing device included in the system. The biological particle sorting device may be as in the 2. described above. Furthermore, the description regarding the information processing unit in the 2. described above applies to the information processing device. In other words, the present disclosure also provides an information processing device including the display unit according to the present disclosure.

An example of the biological particle sorting system according to the present disclosure will be described with reference to FIG. 24. FIG. 24 illustrates a configuration example of a biological particle sorting system of the present disclosure. A biological particle sorting system 400 illustrated in FIG. 24 includes a biological particle sorting device 401 that executes the biological particle sorting processing, and a plurality of information processing devices 402a to 402c configured to be able to operate the biological particle sorting device. The number of information processing devices included in the system is not limited to three as illustrated in FIG. 24, and may be one or more. The plurality of information processing devices is connected to the biological particle sorting device via, for example, a network 403.

The information processing device 402a includes the display unit in the 2. described above. The user sets the sorting processing conditions to be executed in the biological particle sorting device 401 via the display unit. The information processing device 402a illustrated in FIG. 24 operates as the information processing unit 102 in 2. described above. The information processing device 402a causes the display unit attached to or connected to the device to output the processing condition setting screen described in 2. described above. The user sets the sorting processing conditions to be executed in the biological particle sorting device 401 via the screen. In other words, the information processing device 402a receives the input of the sorting processing conditions via the screen. Then, the information processing device 402a causes the biological particle sorting device 401 to execute the sorting processing in accordance with the sorting processing conditions.

Note that the present disclosure can also have the following configurations.

[1]

A biological particle sorting device including:
a display unit that displays a measurement data display area
in which in a display mode that displays blank spaces in addition to one or more pieces of measurement data in the measurement data display area,
in response to a user selecting one piece of movement target measurement data of the one or more pieces of measurement data and one movement destination position in the blank spaces, the movement target measurement data is displayed at the movement destination position; and
after the display position of the movement target measurement data is changed, a position where the movement target measurement data exists is displayed as the blank spaces.
   [2]

The biological particle sorting device according to [1], in which the one or more pieces of measurement data are displayed in a grid pattern in the measurement data display area.

[3]

The biological particle sorting device according to [1] or [2], in which a candidate position image indicating a position where the measurement data can be arranged is displayed in the blank spaces.

[4]

The biological particle sorting device according to any one of [1] to [3], in which an arrangement configuration of measurement data other than the movement target measurement data is not changed between before and after the movement of the movement target measurement data to the blank spaces.

[5]

The biological particle sorting device according to any one of [1] to [4] including:
a plurality of display modes, in which the blank spaces are caused to appear in the measurement data display area in response to selection of a display mode that displays the blank spaces from among the plurality of display modes; or
the blank spaces are caused to appear in the measurement data display area in response to the selection of the display mode that displays the blank spaces from among the plurality of display modes and selection of the movement target measurement data.
   [6]

The biological particle sorting device according to any one of [1] to [5], in which the number of columns of positions where the measurement data can be arranged in the measurement data display area is set in advance.

[7]

The biological particle sorting device according to any one of [1] to [6], in which the biological particle sorting device is configured to be able to display a setting panel that changes the number of columns.

[8]

The biological particle sorting device according to [6] or [7], in which the number of columns is increased in response to the selection of the display mode that displays the blank spaces.

[9]

The biological particle sorting device according to [8], in which the increased columns are displayed as the blank spaces.

[10]

The biological particle sorting device according to any one of [7] to [9], in which the setting panel is displayed translucently.

[11]

The biological particle sorting device according to any one of [1] to [10],
in which the biological particle sorting device is configured to be able to display a gate edit panel that edits a gate set in the measurement data, and
the gate edit panel is configured to be able to select a gate to be edited.
   [12]

The biological particle sorting device according to [11], in which the gate edit panel is configured to be able to select the entire selected gate or a configuration component of the selected gate as an object to be edited.

[13]

The biological particle sorting device according to any one of [1] to [12],
in which the biological particle sorting device is configured to be able to display a plot or axis edit panel that edits a display form and/or axis of the measurement data, and
the plot or axis edit panel is configured to be able to select an axis to be edited.
   [14]

The biological particle sorting device according to any one of [1] to [13],
in which the display unit displays, in addition to the measurement data display area, a sorting operation control area displaying a button that controls a sorting operation by the biological particle sorting device, and
data associated with the sorting operation control area can be expanded and displayed on the measurement data display area.
   [15]

The biological particle sorting device according to any one of [1] to [14],
in which the display unit displays, in addition to the measurement data display area, an operation button area that displays one or more operation buttons used to set sorting conditions, and
the operation button area can be expanded and displayed on the measurement data display area in response to selection of any one of the one or more operation buttons.
   [16]

The biological particle sorting device according to [15], in which a button that selects the display mode that displays the blank spaces is displayed in the operation button area.

[17]

The biological particle sorting device according to any one of [1] to [16], in which the display unit is configured to facilitate touch input.

[18]

A biological particle sorting system including:
a display unit that displays a measurement data display area,
in which in a display mode that displays blank spaces in addition to one or more pieces of measurement data in the measurement data display area,
in response to a user selecting one piece of movement target measurement data of the one or more pieces of measurement data and one movement destination position in the blank spaces, the movement target measurement data is displayed at the movement destination position; and
after the display position of the movement target measurement data is changed, a position where the movement target measurement data exists is displayed as the blank spaces.
   [19]

An information processing device including:
a display unit that displays a measurement data display area,
in which in a display mode that displays blank spaces in addition to one or more pieces of measurement data in the measurement data display area,
in response to a user selecting one piece of movement target measurement data of the one or more pieces of measurement data and one movement destination position in the blank spaces, the movement target measurement data is displayed at the movement destination position; and
after the display position of the movement target measurement data is changed, a position where the movement target measurement data exists is displayed as the blank spaces.

### REFERENCE SIGNS LIST

100 Biological particle sorting device
101 Display unit
102 Information processing unit

1. A biological particle sorting device comprising:
a display unit that displays a measurement data display area,
wherein in a display mode that displays blank spaces in addition to one or more pieces of measurement data in the measurement data display area,
in response to a user selecting one piece of movement target measurement data of the one or more pieces of measurement data and one movement destination position in the blank spaces, the movement target measurement data is displayed at the movement destination position; and
after the display position of the movement target measurement data is changed, a position where the movement target measurement data exists is displayed as the blank spaces.

2. The biological particle sorting device according to claim 1, wherein the one or more pieces of measurement data are displayed in a grid pattern in the measurement data display area.

3. The biological particle sorting device according to claim 1, wherein a candidate position image indicating a position where measurement data can be arranged is displayed in the blank spaces.

4. The biological particle sorting device according to claim 1, wherein an arrangement configuration of measurement data other than the movement target measurement data is not changed between before and after the movement of the movement target measurement data to the blank spaces.

5. The biological particle sorting device according to claim 1, comprising:
a plurality of display modes,
wherein the blank spaces are caused to appear in the measurement data display area in response to selection of a display mode that displays the blank spaces from among the plurality of display modes; or
the blank spaces are caused to appear in the measurement data display area in response to the selection of the display mode that displays the blank spaces from among the plurality of display modes and selection of the movement target measurement data.

6. The biological particle sorting device according to claim 1, wherein the number of columns of positions where the measurement data can be arranged in the measurement data display area is settable.

7. The biological particle sorting device according to claim 6, wherein the biological particle sorting device is configured to be able to display a setting panel that changes the number of columns.

8. The biological particle sorting device according to claim 6, wherein the number of columns is increased in the measurement data display area in response to the selection of the display mode that displays the blank space.

9. The biological particle sorting device according to claim 8, wherein the increased columns are displayed as the blank spaces.

10. The biological particle sorting device according to claim 7, wherein the setting panel is displayed translucently.

11. The biological particle sorting device according to claim 1,
wherein the biological particle sorting device is configured to be able to display a gate edit panel that edits a gate set in the measurement data, and
the gate edit panel is configured to be able to select a gate to be edited.

12. The biological particle sorting device according to claim 11, wherein the gate edit panel is configured to be able to select the entire selected gate or a configuration component of the selected gate as an object to be edited.

13. The biological particle sorting device according to claim 1,
wherein the biological particle sorting device is configured to be able to display a plot or axis edit panel that edits a display form and/or axis of the measurement data, and
the plot or axis edit panel is configured to be able to select an axis to be edited.

14. The biological particle sorting device according to claim 1,
wherein the display unit displays, in addition to the measurement data display area, a sorting operation control area displaying a button that controls a sorting operation by the biological particle sorting device, and
data associated with the sorting operation control area can be expanded and displayed on the measurement data display area.

15. The biological particle sorting device according to claim 1,
wherein the display unit displays, in addition to the measurement data display area, an operation button area that displays one or more operation buttons used to set a sorting condition, and
the operation button area can be expanded and displayed on the measurement data display area in response to selection of any one of the one or more operation buttons.

16. The biological particle sorting device according to claim 15, wherein a button that selects the display mode that displays the blank spaces is displayed in the operation button area.

17. The biological particle sorting device according to claim 1, wherein the display unit is configured to facilitate touch input.

18. A biological particle sorting system comprising:
a display unit that displays a measurement data display area,
wherein in a display mode that displays blank spaces in addition to one or more pieces of measurement data in the measurement data display area,
in response to a user selecting one piece of movement target measurement data of the one or more pieces of measurement data and one movement destination position in the blank spaces, the movement target measurement data is displayed at the movement destination position; and
after the display position of the movement target measurement data is changed, a position where the movement target measurement data exists is displayed as the blank spaces.

19. An information processing device comprising:
a display unit that displays a measurement data display area,
wherein in a display mode that displays blank spaces in addition to one or more pieces of measurement data in the measurement data display area,
in response to a user selecting one piece of movement target measurement data of the one or more pieces of measurement data and one movement destination position in the blank spaces, the movement target measurement data is displayed at the movement destination position; and
after the display position of the movement target measurement data is changed, a position where the movement target measurement data exists is displayed as the blank spaces.
